(19) United States
(12) Patent Application Publication
(10) Pub. No.: US 2023/0364101 A1
(43) Pub. Date: Nov. 16, 2023

(54) DICLOFENAC PRODRUG AND USE OF THE SAME

(71) Applicant: Pumont Biotech Co., Ltd., Tainan City (TW)

(72) Inventors: Jenn-Tsang HWANG, Tainan City (TW); Chien-Wen CHEN, Tainan City (TW); Chun-Hsiung KUEI, Tainan City (TW); Wen-Ling CHEN, Tainan City (TW); Jiaran ZHAO, Tainan City (TW)

(21) Appl. No.: 18/314,750

(22) Filed: May 9, 2023

Related U.S. Application Data
(60) Provisional application No. 63/340,751, filed on May 11, 2022.

(51) Int. Cl. A61K 31/5375; A61K 31/265

(52) U.S. Cl. CPC A61K 31/5375; A61K 31/265; C07B 2200/13

(57) ABSTRACT

Disclosed herein is a diclofenac prodrug represented by formula (I),

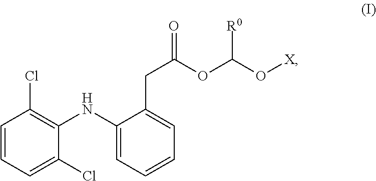

wherein each of the substituents is given the definition as set forth in the Specification and Claims. Also disclosed is a method for alleviating arthritis, which includes administering to a subject in need thereof the aforesaid diclofenac prodrug.

### CROSS-REFERENCE TO RELATED APPLICATION

This application claims the benefit of U.S. Provisional Patent Application No. 63/340,751, filed on May 11, 2022, which is incorporated by reference herein in its entirety.

### FIELD

The present disclosure relates to a diclofenac prodrug and use of the same for alleviating arthritis.

### BACKGROUND

Diclofenac is one of the most commonly prescribed nonsteroidal anti-inflammatory drugs (NSAID) used for treating inflammation and pain induced by inflammation, and is available in various dosage forms suitable for parenteral administration, oral administration, topical administration, etc. The most common side effects of diclofenac are those affecting the gastrointestinal system, which includes abdominal pain, constipation, nausea, ulcers, bleeding, etc.

Systemic administration of diclofenac in high doses (e.g., 150 mg daily) for long term may have adverse effects on the cardiovascular system, and thus, patients diagnosed with congestive heart failure (i.e., NYHA Class II-IV), ischemic heart disease, peripheral artery disease or cerebrovascular disease should not use diclofenac in order to reduce the risk of thromboembolism, while those with cardiovascular risk factors including hypertension, hyperlipidemia, diabetes and those who smoke should only use diclofenac after cautious medical evaluation. Since the risk of cardiovascular disease might increase with increment in dosage and time period of use, diclofenac is recommended to be administered in a minimum effective daily dose for a minimal time period.

It should be noted that when diclofenac is administered parenterally (i.e., injection), the effect of diclofenac cannot be exerted effectively due to fast metabolism thereof, and thus, diclofenac is mainly administered orally. However, oral administration of diclofenac have adverse effects on the gastrointestinal system. In view of the aforesaid, and since the therapeutic benefit of diclofenac still outweighs risk thereof, there is a need for those skilled in the art to develop a diclofenac prodrug that can be administered via injection and that has a slow release effect.

### SUMMARY

Therefore, in a first aspect, the present disclosure provides a diclofenac prodrug which can alleviate at least one of the drawbacks of the prior art.

The diclofenac prodrug is represented by formula (I),

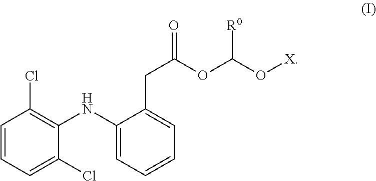

In formula (I), R⁰ represents hydrogen, a straight chain alkyl group or a branched chain alkyl group, X represents R¹,

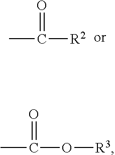

R¹ and R² are independently independently selected from the group consisting of a straight chain alkyl group, a branched chain alkyl group, a substituted heterocyclic group, an unsubstituted heterocyclic group, and

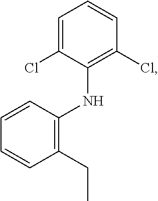 [SMILES: CCc1ccccc1Nc1c(Cl)cccc1Cl]

and R³ is selected from the group consisting of a substituted phenyl group, an unsubstituted phenyl group, a substituted cycloalkyl group, an unsubstituted cycloalkyl group, a substituted heterocyclic group, an unsubstituted heterocyclic group, an alkyl with heterocyclic group, an alkyl with hydroxyl group, and (R³¹O)ₙ—R³², wherein R³¹ represents an alkylene group, R³² represents hydrogen or an alkyl group, and n is an integer greater than 2.

In a second aspect, the present disclosure provides a method for alleviating arthritis which can alleviate at least one of the drawbacks of the prior art.

The method includes administering to a subject in need thereof the aforesaid diclofenac prodrug.

### BRIEF DESCRIPTION OF THE DRAWINGS

Other features and advantages of the present disclosure will become apparent in the following detailed description of the embodiment(s) with reference to the accompanying drawings. It is noted that various features may not be drawn to scale.

FIG. 1 is an X-ray powder diffraction pattern for a diclofenac prodrug of Example 5 (EX5).

FIG. 2 is an X-ray powder diffraction pattern for a diclofenac prodrug of Example 8 (EX8).

FIG. 3 is a graph showing the amount of plasma drug exposure of diclofenac versus time for the rats in the Comparative group (CG), Experimental group 1 (EG1), and Experimental group 2 (EG2).

FIG. 4 is a graph showing the amount of meniscus drug exposure of diclofenac versus time for the rats in the CG, EG1, and EG2.

FIG. 5 is a graph showing the amount of synovial tissue drug exposure of diclofenac versus time for the rats in the CG, EG1, and EG2.

### DETAILED DESCRIPTION

Before the present disclosure is described in greater detail, it should be noted that if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a diclofenac prodrug which is represented by formula (I),

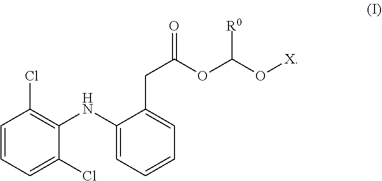

In formula (I), R⁰ represents hydrogen, a straight chain alkyl group or a branched chain alkyl group, X represents R¹,

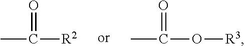

R¹ and R² are independently independently selected from the group consisting of a straight chain alkyl group, a branched chain alkyl group, a substituted heterocyclic group, an unsubstituted heterocyclic group, and

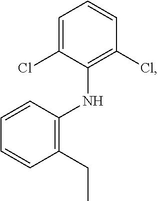 [SMILES: CCc1ccccc1Nc1c(Cl)cccc1Cl]

and R³ is selected from the group consisting of a substituted phenyl group, an unsubstituted phenyl group, a substituted cycloalkyl group, an unsubstituted cycloalkyl group, a substituted heterocyclic group, an unsubstituted heterocyclic group, an alkyl with heterocyclic group, an alkyl with hydroxyl group, and (R³¹O)ₙ—R³², wherein R³¹ represents an alkylene group, R³² represents hydrogen or an alkyl group, and n is an integer greater than 2.

According to the present disclosure, in R¹ and R², an example of the straight chain alkyl group includes C₁-C₈ straight chain alkyl group, an example of the branched chain alkyl group includes C₁-C₈ branched chain alkyl group, and an example of the heterocyclic group includes a nitrogen heterocycle. Examples of the nitrogen heterocycle include saturated 5-membered nitrogen heterocycle and saturated 6-membered nitrogen heterocycle.

According to the present disclosure, in R³, an example of a substituent in the substituted phenyl group includes alkyl, an example of a substituent in the substituted cycloalkyl group includes alkyl, and examples of the heterocyclic group include nitrogen heterocycle and oxygen heterocycle. Examples of the nitrogen heterocycle include saturated 5-membered nitrogen heterocycle and saturated 6-membered nitrogen heterocycle, but are not limited thereto. An example of the oxygen heterocycle include saturated 6-membered nitrogen heterocycle.

According to the present disclosure, in R³, an example of a heterocyclic group in the alkyl with heterocyclic group includes oxygen heterocycle. An example of the alkyl with heterocyclic group includes

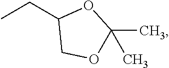

and examples of the alkyl with hydroxyl group includes CH₂CH₂OH, —CH₂CH₂CH₂OH, —CH₂CH(OH)CH₂OH, but are not limited thereto.

According to the present disclosure, in R³¹, examples of the alkylene group include methylene, ethylidine, propylidine, but are not limited thereto.

According to the present disclosure, in R³², examples of the alkyl group include methyl and ethyl.

According to the present disclosure, the diclofenac prodrug of the present disclosure is synthesized according to synthesis pathway (A) illustrated as follows:

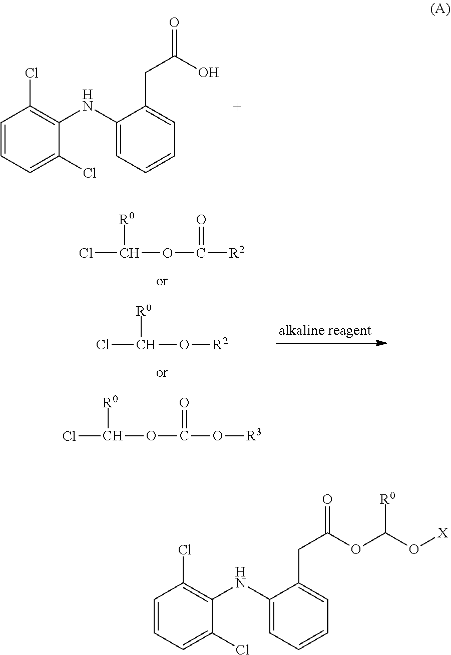

As shown in synthesis pathway (A), diclofenac (i.e., parent drug) is reacted with an alkaline reagent, e.g., N,N-diisopropylethylamine, and then, a substitution reaction is performed in the presence of a solvent, e.g., dimethylformamide.

In certain embodiments, the diclofenac prodrug of the present disclosure is synthesized according to synthesis pathway (B) illustrated as follows:

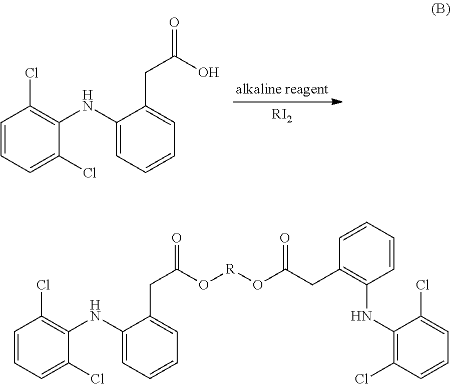

As shown in synthesis pathway (B), diclofenac is reacted with an alkaline reagent, e.g., N,N-diisopropylethylamine, and then reacted with RI₂ in the presence of a solvent, e.g., dimethylformamide, wherein R is CH₂.

According to the present disclosure, an ester carbonate of the diclofenac prodrug of the present disclosure is synthesized according to synthesis pathway (C) illustrated as follows:

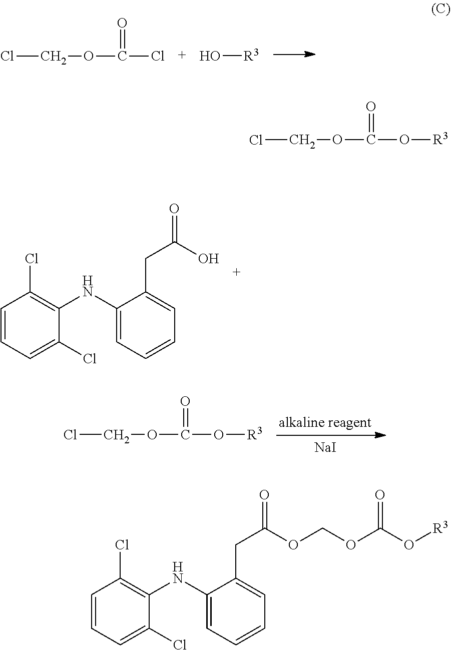

As shown in synthesis pathway (C), chloromethyl chloroformate is reacted with an alcohol represented by HO—R³ in the presence of a solvent, e.g., dichloromethane and/or pyridine to obtain an intermediate product, followed by subjecting the intermediate product and diclofenac to a substitution reaction at 50° C. in the presence of an alkaline solution and sodium iodide, wherein the alkaline solution includes an alkaline reagent (e.g., N,N-diisopropylethylamine) and a solvent (e.g., acetone).

In certain embodiments, an ester urethane of the diclofenac prodrug of the present disclosure is synthesized according to synthesis pathway (D) illustrated as follows:

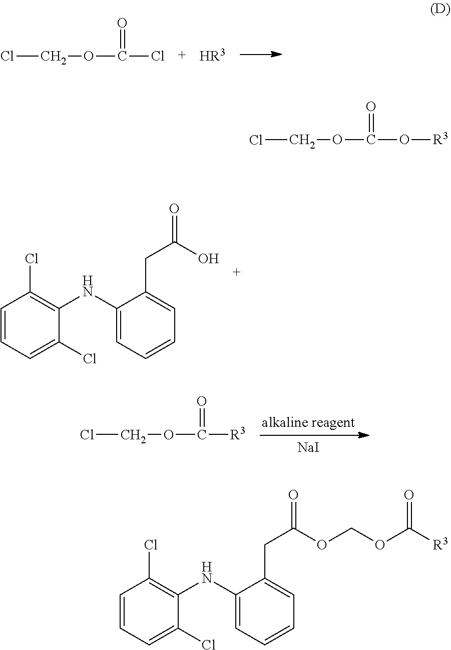

As shown in synthesis pathway (D), chloromethyl chloroformate is reacted with a nitrogen-containing heterocyclic amine represented by HR³ in the presence of a solvent, e.g., dichloromethane and/or pyridine to obtain an intermediate product, followed by subjecting the intermediate product and diclofenac to a substitution reaction at 50° C. in the presence of an alkaline solution and sodium iodide, wherein the alkaline solution includes an alkaline reagent (e.g., N,N-diisopropylethylamine) and a solvent (e.g., acetone).

In certain embodiments, R⁰ represents hydrogen, X represents R¹, and R¹ is selected from the group consisting of C₁-C₈ straight chain alkyl and C₁-C₈ branched chain alkyl.

In certain embodiments, R⁰ represents hydrogen, X represents

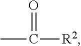

and R² is selected from the group consisting of a straight chain alkyl group, a substituted heterocyclic group, an unsubstituted heterocyclic group, and

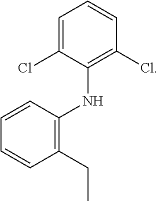 [SMILES: CCc1ccccc1Nc1c(Cl)cccc1Cl]

In certain embodiments, R⁰ represents hydrogen, X represents

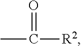

and R² is selected from the group consisting of —CH₂CH₂CH₃,

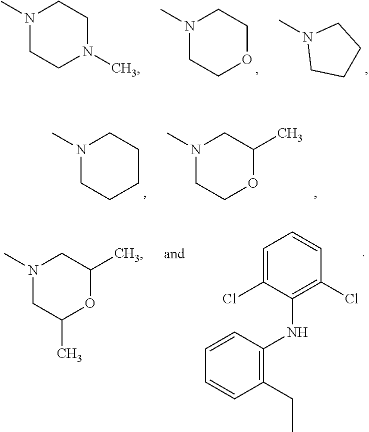

In certain embodiments, R⁰ represents hydrogen, X represents

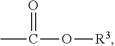

and R³ is selected from the group consisting of

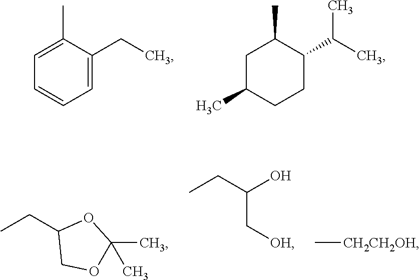

—CH₂CH₂CH₂OH, —CH₂CH₂OCH₂CH₂OH, and CH₂CH₂OCH₂CH₂OCH₃.

In certain embodiments,

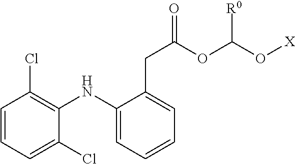

is

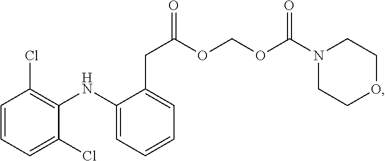 [SMILES: O=C(Cc1ccccc1Nc1c(Cl)cccc1Cl)OCOC(=O)N1CCOCC1]

which is in a crystalline form, and an X-ray diffraction pattern of

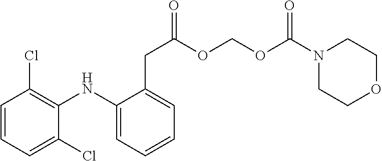 [SMILES: O=C(Cc1ccccc1Nc1c(Cl)cccc1Cl)OCOC(=O)N1CCOCC1]

shows characteristics peaks in 2θ degrees at 13.04, 15.92, 17.14, 18.68, 19.86, 20.35, 21.39, 21.65, 22.42, 23.65, 25.12, 25.68, 28.47 and 28.96.

In certain embodiments,

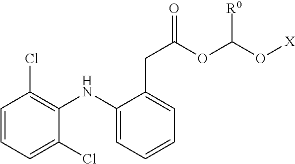

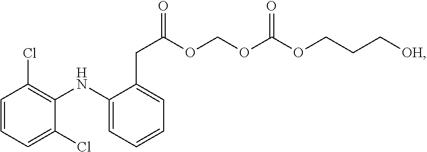 [SMILES: O=C(Cc1ccccc1Nc1c(Cl)cccc1Cl)OCOC(=O)OCCCO]

which is in a crystalline form, and an X-ray diffraction pattern of

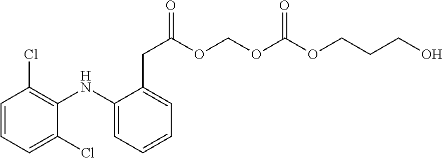 [SMILES: O=C(Cc1ccccc1Nc1c(Cl)cccc1Cl)OCOC(=O)OCCCO]

shows characteristics peaks in 2θ degrees at 9.62, 9.87, 17.96, 19.37, 19.77, 20.35, 21.16, 21.68, 22.15, 24.11, 24.60, 25.70, 27.59, 27.86, 29.17 and 34.96.

The present disclosure also provides a method for alleviating arthritis, which includes administering to a subject in need thereof the aforesaid diclofenac prodrug.

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats.

Examples of the arthritis may include, osteoarthritis, rheumatoid arthritis, gouty arthritis, psoriatic arthritis, and infectious arthritis, but are not limited thereto.

According to the present disclosure, the diclofenac prodrug may be formulated into a dosage form suitable for parenteral administration using technology well known to those skilled in the art.

According to the present disclosure, for parenteral administration, the diclofenac prodrug according to the present disclosure may be formulated into an injection, e.g., a sterile aqueous solution, a dispersion or an emulsion.

The diclofenac prodrug according to the present disclosure may be administered via one of the following parenteral routes: intraperitoneal injection, intrapleural injection, intramuscular injection, intravenous injection, intraarterial injection, intraarticular injection, intrasynovial injection, intrathecal injection, intracranial injection, intraepidermal injection, subcutaneous injection, intradermal injection, and intralesional injection.

In an exemplary embodiment, the diclofenac prodrug is formulated into a dosage form for intraarticular administration.

According to the present disclosure, the diclofenac prodrug formed by chemical bonding is present in a solid crystalline form, and was formed using different combinations of crystallization solvents. The therapeutic effects conferred by the diclofenac prodrug of the present disclosure, when administered by intraarticular injection, may be different from those of the parent drug, i.e., diclofenac. The diclofenac prodrug of the present disclosure, when present in an amorphous form, cannot achieve the desirable effect of quantitative release for long term as that of the parent drug.

According to the present disclosure, the diclofenac prodrug has an average particle size d50 which ranges from 3 μm to 30 μm, so as to avoid irritation to the synovial tissue and meniscus when administered by intraarticular injection. In certain embodiments, the diclofenac prodrug has an average particle size d50 which ranges from 8 μm to 13 μm.

According to the present disclosure, the diclofenac prodrug, when present in a transparent sticky liquid form, may be mixed with an excipient to form a diclofenac prodrug solution. Examples of the excipient may include, Tween 20, Tween 80, polyethylene glycol, and hyaluronic acid, but are not limited thereto. The diclofenac solution may be coated on the inner side of a contact lens, and upon contact with ocular fluid of a subject at pH 7.4, may release the diclofenac prodrug in a dissolved and hydrolyzed form, so as to exert an anti-inflammatory effect, thereby preventing eye discomfort after wear of the contact lens for a long period of time. It should be noted that, diclofenac (i.e., the parent drug), which has poor solubility, can only be coated on the inner side of the contact lens at a relatively low concentration, and cannot have slow or sustained release effect.

The dose and frequency of administration of the diclofenac prodrug may vary depending on the following factors: the severity of the illness or disorder to be treated, routes of administration, and age, physical condition and response of the subject to be treated. In general, the diclofenac prodrug may be administered in a single dose or in several doses.

The present disclosure will be described by way of the following examples. However, it should be understood that the following examples are intended solely for the purpose of illustration and should not be construed as limiting the present disclosure in practice.

### EXAMPLES

### Preparation of Diclofenac Prodrug

### Example 1 (EX1)

The procedures for preparing the diclofenac prodrug of EX1 include the following steps A to D.

In step A, first, 0.586 g (1.98 mmol) of diclofenac was added into a round-bottom flask, and then the diclofenac was dissolved in 4 mL of acetonitrile to obtain a solution. Next, 376 μL of N,N-diisopropylethylamine (2.2 mmol) was added to the solution at room temperature, followed by dropwise adding of 0.268 g (0.995 mmol) of diiodomethane, and stirring overnight to allow a reaction to proceed for 16 hours.

In step B, 1.05 g of diiodomethane was added dropwise to permit the reaction to continue at room temperature for 48 hours, so as to obtain a mixture. During the reaction, high performance liquid chromatography (HPLC) was continuously performed to analyze the contents in the mixture.

In step C, water was added dilute to the mixture, and then dichloromethane was added for twice extraction so as to form an organic layer. The organic layer thus collected was washed with brine once, and then dried using anhydrous sodium sulfate to obtain a dehydrated organic layer.

In step D, the solvent in the dehydrated organic layer was removed under reduced pressure to obtain a crude product. Afterwards, the crude product was subjected to preparative reverse phase HPLC to determine purity thereof. The reverse phase HPLC was performed under the following conditions: GL Science Inertsil ODS-3 (C18) column having a size of 30*250 mm, 10 μm; stepwise gradient elution from 75% acetonitrile/water to 100% acetonitrile; UV detection under a wavelength of 275 nm. The thus collected peak-containing fractions were lyophilized to obtain a diclofenac prodrug of EX1 in the form of a white solid having a weight of 94 mg and a purity of 98% with a yield of 16%.

The diclofenac prodrug of EX1, methylene bis(2-(2-((2,6-dichlorophenyl)amino)phenyl)acetate) with a chemical formula of

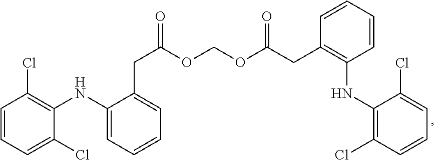 [SMILES: O=C(Cc1ccccc1Nc1c(Cl)cccc1Cl)OCOC(=O)Cc1ccccc1Nc1c(Cl)cccc1Cl]

was subjected to analysis of molecular structures thereof using Varian AS 500 nuclear magnetic resonance (NMR) spectrometer (resonance frequency of protons: 500 MHz for ¹H; 125 MHz for ¹³C). Chemical shifts of the atomic nuclei (in ppm) were recorded relative to those of tetramethylsilane serving as an internal standard.

The diclofenac prodrug of EX1 was determined to have the following characteristics: ¹H NMR (CDCl₃, 500 MHz) δ 3.82 (s, 4H), 5.86 (s, 2H), 6.56 (d, J=8.0 Hz, 2H), 6.65 (s, 2H), 6.93-7.01 (m. 4H), 7.10-7.15 (m, 2H), 7.18-7.22 (m, 2H), 7.33 (d, J=7.5 Hz, 2H); ¹³C NMR (CDCl₃, 125 MHz) δ 38.12, 80.07, 118.61, 122.68, 123.60, 124.08, 128.85, 129.41, 130.97, 137.76, 142.66, 170.93.

### Example 2 (EX2)

The procedures for preparing the diclofenac prodrug of EX2 include the following steps A to E.

In step A, first, 258 mg (176 μL, 2 mmol) of diclofenac, 244 mg (2 mmol) of 2-ethylphenol, and 4 mL of dichloromethane was mixed to form a mixture, which was cooled to 0° C. Next, 210 μL of pyridine (2.6 mmol) was added dropwise to the cooled mixture, followed by stirring at 0° C. for 2 hours and then at room temperature overnight. Thereafter, an extraction treatment was performed by adding water and dichloromethane, followed by collecting a resultant organic layer. The organic layer was washed with sodium bicarbonate and brine, and then dried using anhydrous sodium sulfate, followed by a filtration treatment and a concentration treatment, thereby obtaining chloromethyl (2-ethylphenyl) carbonate in the form a brown oil having a weight of 0.36 g. The chloromethyl (2-ethylphenyl) carbonate was subjected to NMR spectrometry, and ¹H NMR (CDCl₃, 500 MHz) spectrum thereof had the following characteristics: ¹H NMR (CDCl₃, 500 MHz) δ 1.21-1.26 (m, 3H), 2.59-2.64 (m, 2H), 5.93 (s, 2H), 7.16-7.37 (m, 4H).

In step B, 0.32 g (1.08 mmol) of diclofenac was added into a round-bottom flask, and then the round-bottom flask was placed in an ice bath having a temperature ranging from 0° C. to 3° C. Next, 4 mL of N,N-dimethylacetamide was added to dissolve the diclofenac, followed by adding 369 mg of N,N-diisopropylethylamine (2.16 mmol) dropwise, and 0.36 g (1.08 mmol) of chloromethyl (2-ethylphenyl) carbonate, so as to obtain a mixture.

In step C, the mixture was stirred to permit a reaction to continue at room temperature for 72 hours. During the reaction, HPLC was continuously performed to analyze the contents in the mixture.

In step D, water was added to dilute the mixture, and then dichloromethane was added for twice extraction so as to form an organic layer. The organic layer thus collected was washed with brine once, and then dried using anhydrous sodium sulfate to obtain a dehydrated organic layer.

In step E, the solvent in the dehydrated organic layer was removed under reduced pressure to obtain a crude product. Afterwards, the crude product was subjected to preparative LC to determine purity thereof. The reverse phase HPLC was performed under the following conditions: GL Science Inertsil ODS-3 (C18) column having a size of 30*250 mm, 10 μm; isocratic elution using 85% acetonitrile; UV detection under a wavelength of 220 nm. The thus collected peak-containing fractions were lyophilized to obtain a diclofenac prodrug of EX2 in the form of a yellow powder having a weight of 134 mg and a purity of 98.2% with a yield of 26%.

The diclofenac prodrug of EX2, (((2-ethylphenoxy)carbonyl)oxy)methyl 2-(2-((2,6-dichlorophenyl)-amino)phenyl)acetate with a chemical formula of

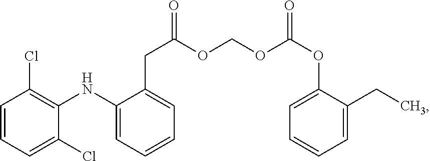 [SMILES: CCc1ccccc1OC(=O)OCOC(=O)Cc1ccccc1Nc1c(Cl)cccc1Cl]

was subjected to NMR spectrometry using the procedures as described in EX1.

The diclofenac prodrug of EX2 was determined to have the following characteristics: ¹H NMR (CDCl₃, 500 MHz) δ 1.18 (t, J=7.5 Hz, 3H), 2.57 (q, J=7.5 Hz, 2H), 3.93 (s, 2H), 5.93 (s, 2H), 6.58-6.60 (m, 2H), 6.67 (s, 1H), 6.98-7.02 (m, 2H), 7.09-7.27 (m, 5H), 7.35 (d, J=8.0 Hz, 2H); ¹³C NMR (CDCl₃, 125 MHz) δ 14.15, 22.89, 38.12, 82.73, 118.69, 121.44, 122.36, 123.53, 124.15, 126.77, 127.00, 128.36, 128.87, 129.45, 129.63, 130.98, 135.58, 137.75, 142.70, 148.82, 152.53, 170.73.

### Example 3 (EX3)

The procedures for preparing the diclofenac prodrug of EX3 include the following steps A to E.

In step A, first, 265 mg (3 mmol) of chloromethylchloroformate, 477 mg (3.05 mmol) of menthol, and 4 mL of dichloromethane was mixed to form a first mixture, which was cooled to 0° C. Next, 291 μL of pyridine (3.05 mmol) was added dropwise to the cooled first mixture, followed by stirring at 0° C. for 2 hours and then at room temperature overnight. Thereafter, an extraction treatment was performed by adding water and dichloromethane, followed by collecting a resultant organic layer. The organic layer was washed with sodium bicarbonate and brine, and then dried using anhydrous magnesium sulfate, followed by a filtration treatment and a concentration treatment, thereby obtaining chloromethyl ((1R,2S,5R)-2-isopropyl-5-methylcyclohexyl, M-CI) carbonate in the form a brown oil having a weight of 0.678 g. The chloromethyl ((1R,2S,5R)-2-isopropyl-5-methylcyclohexyl, M-CI) carbonate was subjected to NMR spectrometry, and ¹H NMR (CDCl₃, 500 MHz) spectrum thereof had the following characteristics: ¹H NMR (CDCl₃, 500 MHz) δ 0.79 (d, J=7.0 Hz, 3H), 0.86-0.93 (m, 8H), 1.03-1.12 (m, 2H), 1.58-1.60 (m, 1H), 1.68-1.71 (m, 2H), 1.88-1.96 (m, 1H), 2.10-2.14 (m, 1H), 4.57-4.61 (m, 1H), 5.71-5.75 (m, 2H).

In step B, 0.681 g (2.3 mmol) of diclofenac was added into a round-bottom flask, and then 8 mL of acetone was added at room temperature to dissolve the diclofenac, followed by adding 418 μL (2.4 mmol) of N,N-diisopropylethylamine dropwise, 3.5 mL of a solution containing 0.678 g (2.72 mmol) of chloromethyl ((1R,2S,5R)-2-isopropyl-5-methylcyclohexyl) carbonate and acetone, and 100 mg of sodium iodide, so as to obtain a second mixture.

In step C, the second mixture was stirred to permit a reaction to continue at room temperature for 16 hours, and the thus formed reactant was heated and stirred at a temperature ranging from 50° C. to 53° C. for 8 hours. During the reaction, HPLC was continuously performed to analyze the contents in the reactant.

In step D, water was added to dilute the mixture, and then dichloromethane was added for twice extraction so as to form an organic layer. The organic layer thus collected was washed with brine once, and then dried using anhydrous sodium sulfate to obtain a dehydrated organic layer.

In step E, the solvent in the dehydrated organic layer was removed under reduced pressure to obtain a crude product. Afterwards, the crude product was subjected to fast silica gel chromatography to determine purity thereof. The gel chromatography was performed under the following conditions: silica gel (Merck) having a size of 40 to 63 μm, 60 Å; and elution using hexane and ethyl acetate in a volume ratio of 1:10. The thus collected eluate fractions were lyophilized to obtain a diclofenac prodrug of EX3 having a weight of 1.06 g and a purity of 98.9% with a yield of 90.7%.

The diclofenac prodrug of EX3, (((((1R,2S,5R)-2-isopropyl-5-methylcyclohexyl)oxy)carbonyl)oxy)-methyl 2-(2-((2,6-dichlorophenyl)amino)phenyl)acetate with a chemical formula of

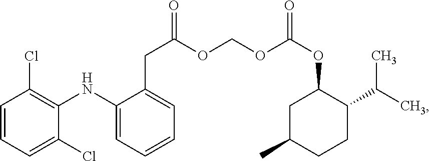 [SMILES: CC(C)[C@@H]1CC[C@@H](C)C[C@H]1OC(=O)OCOC(=O)Cc1ccccc1Nc1c(Cl)cccc1Cl]

was subjected to NMR spectrometry using the procedures as described in EX1.

The diclofenac prodrug of EX3 was determined to have the following characteristics: ¹H NMR (CDCl₃, 500 MHz) δ 0.79 (d, J=7.0 Hz, 3H), 0.82-0.94 (m, 7H), 1.01-1.11 (m, 2H), 1.38-1.57 (m, 2H), 1.67-1.69 (m, 2H), 1.90-1.97 (m, 1H), 2.08-2.11 (m, 1H), 3.88 (s, 2H), 4.53-4.57 (m, 1H), 5.79-5.83 (m, 2H), 6.56 (d, J=7.5 Hz, 1H), 6.69 (s, 1H), 6.97-7.01 (m, 2H), 7.15-7.23 (m, 1H), 7.24-7.26 (m, 1H), 7.34 (d, J=8.0 Hz, 2H).

### Example 4 (EX4)

The procedures for preparing the diclofenac prodrug of EX4 include the following steps A to E.

In step A, a first solution containing 265 μL (3 mmol) of chloromethylchloroformate and 4 mL of dichloromethane was cooled to 0° C., and then 477 mg (3.05 mmol) of 1-methylpiperazine was added dropwise to form a first mixture. The first mixture was stirred at 0° C. for 2 hours and then at room temperature overnight, followed by a concentration treatment to obtain chloromethyl 4-methylpiperazine-1-carboxylate in the form a powdered solid having a weight of 0.678 g. The chloromethyl 4-methylpiperazine-1-carboxylate was subjected to NMR spectrometry, and ¹H NMR (CDCl₃, 500 MHz) spectrum thereof had the following characteristics: ¹H NMR (CDCl₃, 500 MHz) δ 2.94 (s, 2H), 3.11-3.64 (m, 6H), 4.21-4.38 (m, 2H), 5.86 (s, 2H).

In step B, 430 mg (1.45 mmol) of diclofenac was added into a round-bottom flask, and then 5 mL of acetone was added at room temperature to dissolve the diclofenac, followed by adding 333 mg (1.45 mmol) of chloromethyl 4-methylpiperazine-1-carboxylate dissolved in 2 mL of acetone, and 50 mg (0.33 mmol) of sodium iodide, so as to obtain a second mixture.

In step C, the second mixture was heated and stirred at 53° C. for 16 hours, followed by a concentration treatment under reduced pressure to obtain a concentrated product.

In step D, the concentrated product was diluted by adding 30 mL of water, and then dichloromethane was added for twice extraction (20 mL each time) so as to form an organic layer. The organic layer thus collected was washed with 20 mL of brine, and then dried using anhydrous magnesium sulfate, followed by a filtration treatment so as to obtain a dehydrated organic layer.

In step E, the solvent in the dehydrated organic layer was removed under reduced pressure to obtain a crude product, which was diluted using 4.0 mL of acetonitrile/water solvent and then subjected to preparative reverse phase HPLC to determine purity thereof. The reverse phase HPLC was performed under the following conditions: GL Science Inertsil ODS-3 (C18) column having a size of 30*250 mm, 10 μm; flow rate of 32.5 mL/minute; stepwise gradient elution from 43% acetonitrile/water to 90% acetonitrile/water with 0.1% of trifluoroacetic acid in water; UV detection under a wavelength of 245 nm. The thus collected peak-containing fractions were lyophilized to obtain a diclofenac prodrug of EX4 in the form of a white solid having a weight of 296 mg and a purity of 98% with a yield of 45.2%.

The diclofenac prodrug of EX4, (2-(2-((2,6-dichlorophenyl)amino)phenyl)acetoxy)methyl 4-methylpiperazine-1-carboxylate with a chemical formula of

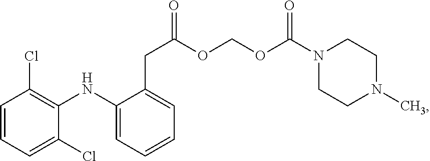 [SMILES: CN1CCN(C(=O)OCOC(=O)Cc2ccccc2Nc2c(Cl)cccc2Cl)CC1]

was subjected to NMR spectrometry using the procedures as described in EX1.

The diclofenac prodrug of EX4 was determined to have the following characteristics: ¹H NMR (CDCl₃, 500 MHz) δ 2.61-2.74 (m, 2H), 2.75 (s, 3H), 3.25-3.61 (m, 4H), 3.06 (s, 2H), 3.90-4.31 (m, 2H), 5.82 (br, s, 2H), 6.52-6.59 (m, H), 6.65 (br, s, 1H), 6.95-7.04 (m, 2H), 7.13-7.16 (m, 1H), 7.22-7.29 (m, 1H), 7.35 (d, J=8.5 Hz, 2H); ¹³C NMR (CDCl₃, 125 MHz) δ 38.23, 41.15, 53.03, 80.99, 118.29, 122.16, 123.44, 124.47, 128.38, 128.96, 129.55, 131.05, 142.73, 152.66, 171.16.

### Example 5 (EX5)

The procedures for preparing the diclofenac prodrug of EX5 include the following steps A to E.

In step A, a first solution containing 176 μL (2 mmol) of chloromethylchloroformate and 2 mL of dichloromethane was cooled to 0° C., and then mixed with a second solution containing 174 mg (2 mmol) of morphine, 210 μL of pyridine, and 2 mL of dichloromethane to form a first mixture. The first mixture was stirred at 0° C. for 2 hours and then at room temperature overnight, followed by a concentration treatment to obtain chloromethyl morpholine-4-carboxylate in the form of an intermediate oily product which had a weight of 272 mg with a yield of 70%, and which may be further subjected to a freeze-drying process to obtain an amorphous product. The chloromethyl morpholine-4-carboxylate was subjected to NMR spectrometry, and ¹H NMR (CDCl₃, 500 MHz) spectrum thereof had the following characteristics: ¹H NMR (CDCl₃, 500 MHz) δ 3.52 (br s, 4H), 3.64-3.74 (m, 4H), 5.80 (s, 2H).

In step B, 418 mg (1.41 mmol) of diclofenac dissolved in 4 mL of acetone was mixed with 260 μL (1.5 mmol) of N,N-diisopropylethylamine, and then 272 mg (1.45 mmol) of the chloromethyl morpholine-4-carboxylate dissolved in 2 mL of acetone and 100 mg (0.67 mmol) of sodium iodide were added in small portions to form a second mixture.

In step C, the second mixture was heated and stirred at 53° C. for 16 hours, followed by a concentration treatment under reduced pressure to obtain a concentrated product.

In step D, the concentrated product was diluted by adding 30 mL of water, and then dichloromethane was added for twice extraction (20 mL each time) so as to form an organic layer. The organic layer thus collected was washed with 20 mL of brine, and then dried using anhydrous magnesium sulfate, followed by a filtration treatment so as to obtain a dehydrated organic layer.

In step E, the solvent in the dehydrated organic layer was removed under reduced pressure to obtain a crude product, which was diluted using 4.0 mL of acetonitrile/water solvent and then subjected to preparative reverse phase HPLC to determine purity thereof. The preparative reverse phase HPLC was performed under the following conditions: GL Science Inertsil ODS-3 column having a size of 30*250 mm, 10 μm; flow rate of 32.5 mL/minute; stepwise gradient elution from 60% acetonitrile/water to 74% acetonitrile/water with 0.1% of trifluoroacetic acid in water; UV detection under a wavelength of 254 nm. The thus collected peak-containing fractions were lyophilized to obtain a diclofenac prodrug of EX5 in the form of a crystalline solid having a weight of 124 mg and a purity of 95% with a yield of 34.5%.

The diclofenac prodrug of EX5, (2-(2-((2,6-dichlorophenyl)amino)phenyl)acetoxy)methyl morpholine-4-carboxylate with a chemical formula of

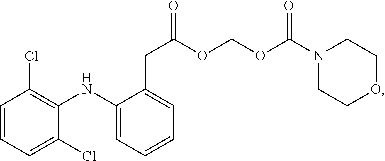 [SMILES: O=C(Cc1ccccc1Nc1c(Cl)cccc1Cl)OCOC(=O)N1CCOCC1]

was subjected to NMR spectrometry using the procedures as described in EX1.

The diclofenac prodrug of EX5 was determined to have the following characteristics: ¹H NMR (CDCl₃, 500 MHz) δ 3.41-3.65 (m, 8H), 3.87 (br s, 2H), 5.83 (br s, 2H), 6.56 (d, J=8.0 Hz, 1H), 6.71 (bs, 1H), 6.95-7.12 (m, 2H), 7.14-7.16 (m, 2H), 7.23-7.25 (m, 1H), 7.34 (d, J=8.0 Hz, 2H); ¹³C NMR (CDCl₃, 125 Hz) δ 38.30, 43.96, 44.40, 66.29, 66.48, 80.76, (C, —OCH₂O—), 118.47, 122.21, 123.69, 124.13, 128.23, 128.87, 129.47, 130.98, 137.74, 142.69, 153.36 (C, —OCOO—), 171.27 (C, —OCOCH₂—).

### Example 6 (EX6)

The procedures for preparing the diclofenac prodrug of EX6 include the following steps A to E.

In step A, a first solution containing 180 μL (2.05 mmol) of chloromethylchloroformate and 2 mL of dichloromethane was cooled to 0° C., and then mixed with a second solution containing 274 mg (2 mmol) of (S)-(+)2,2-dimethyl-1,3-dioxolane-4-methanol, 210 μL of pyridine, and 3 mL of dichloromethane to form a first mixture. Next, the first mixture was stirred at 0° C. for 2 hours and then at room temperature overnight, followed by adding water and dichloromethane for extraction so as to form an organic layer. Thereafter, the organic layer thus formed was washed with brine, and then dried using anhydrous magnesium sulfate, followed by a filtration treatment so as to obtain a dehydrated organic layer. Afterwards, the dehydrated organic layer was subjected to a concentration treatment so as to obtain chloromethyl ((2,2-dimethyl-1,3-dioxolan-4-yl)methyl) carbonate in the form of an intermediate oily product having a weight of 363 mg with a yield of 81%. The chloromethyl ((2,2-dimethyl-1,3-dioxolan-4-yl)methyl) carbonate was subjected to NMR spectrometry, and ¹H NMR (CDCl₃, 500 MHz) spectrum thereof had the following characteristics: ¹H NMR (CDCl₃, 500 MHz) δ 1.36 (s, 3H), 1.44 (s, 3H), 3.78-3.81 (m, 1H), 4.09-4.11 (m, 1H), 4.21-4.29 (m, 2H), 4.34-4.36 (m, 1H), 5.72-5.75 (m, 2H).

In step B, 280 μL (1.63 mmol) of N,N-diisopropylethylamine was added to a third solution containing 478 mg (1.61 mmol) of diclofenac dissolved in 4 mL of acetone, followed by adding 363 mg (1.45 mmol) of chloromethyl ((2,2-dimethyl-1,3-dioxolan-4-yl)methyl) carbonate dissolved in 2 mL of acetone, and 100 mg (0.67 mmol) of sodium iodide, so as to obtain a second mixture.

In step C, the second mixture was heated and stirred at 53° C. for 24 hours, followed by a concentration treatment under reduced pressure to obtain a concentrated product.

In step D, the concentrated product was diluted by adding 30 mL of water, and then dichloromethane was added for twice extraction (20 mL each time) so as to form an organic layer. The organic layer thus collected was washed with 20 mL of brine, and then dried using anhydrous magnesium sulfate, followed by a filtration treatment so as to obtain a dehydrated organic layer.

In step E, the solvent in the dehydrated organic layer was removed under reduced pressure to obtain a crude product, which was diluted using 4.0 mL of acetonitrile/water solvent and then subjected to preparative reverse phase HPLC to determine purity thereof. The preparative reverse phase HPLC was performed under the following conditions: GL Science Inertsil ODS-3 column having a size of 30*250 mm, 10 μm; flow rate of 32.5 mL/minute; stepwise gradient elution from 60% acetonitrile/water to 74% acetonitrile/water with 0.1% of trifluoroacetic acid in water; UV detection under a wavelength of 275 nm. The thus collected peak-containing fractions were lyophilized to obtain a diclofenac prodrug of EX6 in the form of a colorless oil having a weight of 417 mg and a purity of 95% with a yield of 53.5%. It should be noted that the colorless oil may be further subjected to a recrystallization treatment using ethyl acetate and n-hexane that are present in a volume ratio ranging from 1:6 to 1:8 so as to obtain a crystalline product.

The diclofenac prodrug of EX6, ((((2,2-dimethyl-1,3-dioxolan-4-yl)methoxy)carbonyl)oxy)methyl 2-(2-((2,6-dichlorophenyl)amino)phenyl)acetate with a chemical formula of

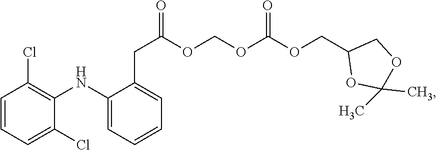 [SMILES: CC1(C)OCC(COC(=O)OCOC(=O)Cc2ccccc2Nc2c(Cl)cccc2Cl)O1]

was subjected to NMR spectrometry using the procedures as described in EX1.

The diclofenac prodrug of EX6 was determined to have the following characteristics: ¹H NMR (CDCl₃, 500 MHz) δ 1.35 (s, 3H), 1.42 (s, 3H), 3.77-3.79 (m, 1H), 3.88 (s, 2H), 4.05-4.07 (m, 1H), 4.20 (br s, 2H), 4.32-4.34 (m, 1H), 5.81 (s, 2H), 6.56 (d, J=8.0 Hz, 1H), 6.62 (br s, 1H), 6.95-6.99 (m, 2H), 7.12-7.15 (m, 1H), 7.12-7.15 (m, 1H), 7.2-7.25 (m, 1H), 7.34 (d, J=8.5 Hz, 2H); ¹³C NMR (CDCl₃, 125 Hz) δ 25.29, 26.64, 38.09, 66.15, 68.42, 73.02, 82.44, 110.11, 118.65, 122.30, 123.55, 124.11, 128.31, 129.44, 130.98, 137.75, 142.68, 153.72, 170.69.

### Example 7 (EX7)

The procedures for preparing the diclofenac prodrug of EX7 include the following steps A to C.

In step A, 0.8 mL of trifluoroacetic acid (10.7 mmol) was added dropwise at 25° C. to a solution containing ((((2,2-dimethyl-1,3-dioxolan-4-yl)methoxy)carbonyl)oxy)methyl 2-(2-((2,6-dichlorophenyl)amino)phenyl)acetate as described in Example 6, 15 mL of methanol, and 3 mL of deionized water, so as to form a mixture.

In step B, the mixture was stirred to permit a reaction to continue at room temperature for 16 hours. After completeness of the reaction was confirmed by reverse phase HPLC, the mixture was evaporated to obtain a dry product.

In step C, the dry product was diluted using 4.0 mL of acetonitrile/water solvent and then subjected to preparative reverse phase HPLC to determine purity thereof. The preparative reverse phase HPLC was performed under the following conditions: GL Science Inertsil ODS-3 column having a size of 30*250 mm, 10 μm; flow rate of 32.5 mL/minute; stepwise gradient elution from 43% acetonitrile/water to 70% acetonitrile/water with 0.1% of trifluoroacetic acid in water; UV detection under a wavelength of 254 nm. The thus collected peak-containing fractions were lyophilized to obtain a diclofenac prodrug of EX7 in the form of a colorless oil having a weight of 198 mg and a purity of 96% with a yield of 58.9%. It should be noted that the colorless oil may be further subjected to a recrystallization treatment using ethyl acetate and n-hexane that are present in a volume ratio ranging from 1:5 to 1:8 so as to obtain a crystalline product.

The diclofenac prodrug of EX7, (((2,3-dihydroxypropoxy)carbonyl)oxy)methyl 2-(2-((2,6-dichlorophenyl)amino)phenyl)acetate with a chemical formula of

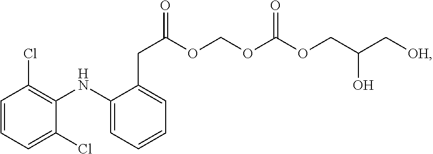 [SMILES: O=C(Cc1ccccc1Nc1c(Cl)cccc1Cl)OCOC(=O)OCC(O)CO]

was subjected to NMR spectrometry using the procedures as described in EX1.

The diclofenac prodrug of EX7 was determined to have the following characteristics: ¹H NMR (CDCl₃, 500 MHz) δ 3.33 (br s, 3H), 3.54-3.56 (m, 1H), 3.66 (br s, 3H), 3.86 (s, 2H), 3.92 (br s, 1H), 4.16-4.23 (m, 2H), 4.16-4.23 (m, 2H), 5.78 (s, 2H), 6.54 (d, J=7.5 Hz, 1H), 6.58 (br s, 1H), 6.94-6.97 (m, 2H), 7.10-7.13 (m, 1H), 7.20-7.22 (m, 1H), 7.32 (d, J=8.5 Hz, 2H); ¹³C NMR (CDCl₃, 125 Hz) δ 38.02, 63.03, 69.24, 69.71, 82.56, 118.55, 122.29, 123.46, 124.24, 128.35, 128.89, 129.48, 131.03, 137.69, 142.65, 153.90, 170.97.

### Example 8 (EX8)

The procedures for preparing the diclofenac prodrug of EX8 include the following steps A to E.

In step A, a first solution containing 771 μL (8.5 mmol) of chloromethylchloroformate and 10 mL of dichloromethane was cooled to 0° C., and then mixed with a second solution containing 1.52 (8 mmol) of 3-((tert-butyldimethylsilyl)oxy)-propanol, 683 μL of pyridine, and 4 mL of dichloromethane to form a first mixture. Next, the first mixture was stirred at 0° C. for 2 hours and then at room temperature overnight, and then subjected to a first extraction treatment by adding 20 mL of water and 20 mL of dichloromethane. The resultant aqueous layer and organic layer were collected separately, and then 20 mL of dichloromethane was added to the aqueous layer. Thereafter, the aqueous layer added with the dichloromethane was subjected to a second extraction treatment, and the resultant organic layer was collected. The organic layers thus collected from the first and second extractions were combined, and then washed with 20 mL of brine, and then dried using anhydrous sodium sulfate, followed by a filtration treatment so as to obtain a dehydrated organic layer. Afterwards, the dehydrated organic layer was subjected to an evaporation treatment and a concentration treatment under reduced pressure, so as to obtain 3-((tert-butyldimethylsilyl)oxy)propan-1-ol in the form of an intermediate oily product having a weight of 2.17 g with a yield of 96%. The 3-((tert-butyldimethylsilyl)oxy)propan-1-ol was subjected to NMR spectrometry, and ¹H NMR (CDCl₃, 500 MHz) spectrum thereof had the following characteristics: ¹H NMR (CDCl₃, 500 MHz) δ 0.5 (s, 6H), 0.89 (s, 9H), 1.88-1.92 (m, 2H), 3.72 (t, J=6.0 Hz, 2H), 4.34 (t, J=6.5 Hz, 2H), 5.73 (s, 2H).

In step B, 2.16 g (7.3 mmol) of diclofenac dissolved in 20 mL of acetone was mixed with 1.31 mL (7.3 mmol) of N,N-diisopropylethylamine under nitrogen atmosphere, and then 2.16 g (7.69 mmol) of the 3-((tert-butyldimethylsilyl)oxy)propan-1-ol dissolved in 40 mL of acetone and 100 mg (0.67 mmol) of sodium iodide were added to form a second mixture.

In step C, the second mixture was stirred at 65° C. for 26 hours, and then subjected to a concentration treatment by evaporation to remove solvent therein, so as to obtain a concentrated product. Thereafter, the concentrated product was mixed with 30 mL of isopropyl ether, stirred for 30 minutes, and then filtered and concentrated to obtain an oily crude product.

In step D, the oily crude product was subjected to flash chromatography using hexane and ethyl acetate in a volume ratio of 10:1 so as to determine purity thereof. The thus collected eluate fractions were lyophilized to obtain 9,9,10,10-tetramethyl-3-oxo-2,4,8-trioxa-9-silaundecyl 2-(2-((2,6-dichlorophenyl)amino)phenyl)acetate having a weight of 1.62 g with a yield of 41%.

The 9,9,10,10-tetramethyl-3-oxo-2,4,8-trioxa-9-silaundecyl 2-(2-((2,6-dichlorophenyl)amino)phenyl)acetate was subjected to NMR spectrometry using the procedures as described in EX1, and had the following characteristics: ¹H NMR (CDCl₃, 500 MHz) δ 0.01 (s, 6H), 0.89 (s, 9H), 1.86-1.90 (m, 2H), 3.69 (t, J=6.0 Hz, 2H), 3.88 (s, 2H), 4.29 (t, J=6.5, 2H), 5.81 (s, 2H), 6.57 (d, J=8.0 Hz, 1H), 6.64 (bs, 1H), 6.96-6.99 (m, 2H), 7.12-7.15 (m, 2H), 7.12-7.15 (m, 1H), 7.23 (bs, 1H), 7.34 (d, J=8.0 Hz, 2H); ¹³C NMR (CDCl₃, 125 Hz) δ 25.64, 31.53, 38.13, 58.91, 65.85, 82.30, (C, —OCH₂O—), 118.64, 122.28, 123.63, 124.07, 128.27, 128.84, 129.44, 130.96, 137.78, 142.68, 153.86 (C, —OCOO—), 170.78 (C, —OCOCH₂—).

In step E, 1 mL of trifluoroacetic acid was added at 0° C. to a solution containing 1.62 g (2.99 mmol) of 9,9,10,10-tetramethyl-3-oxo-2,4,8-trioxa-9-silaundecyl 2-(2-((2,6-dichlorophenyl)amino)phenyl)acetate and 16 mL of acetonitrile, so as to form a mixture. Thereafter, the mixture was warmed to 18° C. and left standing for 3 hours. The resultant reactant was directly purified by preparative HPLC under the following conditions: stepwise gradient elution from 50% acetonitrile/water to 75% acetonitrile/water with 0.1% of trifluoroacetic acid in water. The thus collected peak-containing fractions were lyophilized to obtain a diclofenac prodrug of EX8 in the form of a white crystalline solid having a weight of 1.16 g and a purity of 99% with a yield of 91%.

The diclofenac prodrug of EX8, (((3-hydroxypropoxy)carbonyl)oxy)methyl 2-(2-((2,6-dichlorophenyl)amino)phenyl)acetate with a chemical formula of

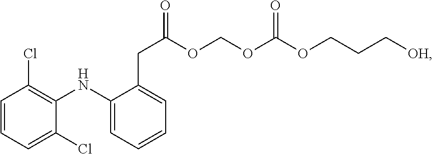 [SMILES: O=C(Cc1ccccc1Nc1c(Cl)cccc1Cl)OCOC(=O)OCCCO]

was subjected to NMR spectrometry using the procedures as described in EX1.

The diclofenac prodrug of EX8 was determined to have the following characteristics: ¹H NMR (CDCl₃, 500 MHz) δ 1.70 (s, 2H), 1.89-1.93 (m, 2H), 3.72 (t, J=6.0 Hz, 2H), 3.88 (s, 2H), 4.34 (t, J=6.5 Hz, 2H), 5.81 (s, 2H), 6.56 (d, J=8.0 Hz, 1H), 6.56 (d, J=8.0 Hz, 1H), 6.64 (bs, 1H), 6.96-6.99 (m, 2H), 7.12-7.15 (m, 2H), 7.12-7.15 (m, 2H), 7.23 (bs, 1H), 7.34 (d, J=8.0 Hz, 2H); ¹³C NMR (CDCl₃, 125 Hz) δ 31.41, 38.11, 58.84, 65.77, 82.38, (C, —OCH₂O—), 118.63, 122.29, 123.57, 124.13, 128.29, 129.44, 130.98, 137.75, 142.68, 154.05 (C, —OCOO—), 170.78 (C, —OCOCH₂—).

### Example 9 (EX9)

The procedures for preparing the diclofenac prodrug of EX9 include the following steps A to E.

In step A, a first solution containing 185 μL (2 mmol) of chloromethylchloroformate and 2 mL of dichloromethane was cooled to 0° C., and then mixed with a second solution containing 318 mg (3 mmol) of diethylene glycol, 210 μL of pyridine, and 3 mL of dichloromethane to form a first mixture. Next, the first mixture was stirred at 0° C. for 2 hours and then at room temperature overnight, followed by adding 30 mL of water and dichloromethane for extraction that is performed twice so as to form an organic layer. Thereafter, the organic layer thus collected was dried using anhydrous magnesium sulfate, followed by a filtration treatment so as to obtain a dehydrated organic layer. Afterwards, the dehydrated organic layer was subjected to a concentration treatment so as to obtain (((2-(2-hydroxyethoxy)ethoxy)carbonyl)oxy)methyl chloride in the form of an intermediate oily product having a weight of 286 mg with a yield of 72%.

In step B, 426 mg (1.44 mmol) of diclofenac dissolved in 4.5 mL of acetone was mixed with 275 μL (1.44 mmol) of N,N-diisopropylethylamine, and then 286 mg (1.44 mmol) of the (((2-(2-hydroxyethoxy)ethoxy)carbonyl)oxy)methyl chloride dissolved in 2 mL of acetone, and 216 mg (1.44 mmol) of sodium iodide were added in small portions, so as to form a second mixture.

In step C, the second mixture was heated and stirred at 55° C. for 24 hours, followed by a concentration treatment under reduced pressure to obtain a concentrated product.

In step D, the concentrated product was diluted by adding 30 mL of water, and then dichloromethane was added for twice extraction (20 mL each time) so as to form an organic layer. The organic layer thus collected was washed with 20 mL of brine, and then dried using anhydrous magnesium sulfate, followed by a filtration treatment so as to obtain a dehydrated organic layer.

In step E, the solvent in the dehydrated organic layer was removed under reduced pressure to obtain a crude product, which was diluted using 4.0 mL of acetonitrile/water solvent and then subjected to preparative reverse phase HPLC to determine purity thereof. The preparative reverse phase HPLC was performed under the following conditions: GL Science Inertsil ODS-3 column having a size of 30*250 mm, 10 μm; flow rate of 32.5 mL/minute; stepwise gradient elution from 55% acetonitrile/water to 60% acetonitrile/water with 0.1% of trifluoroacetic acid in water; UV detection under a wavelength of 275 nm. The thus collected peak-containing fractions were lyophilized to obtain a diclofenac prodrug of EX9 in the form of a colorless oil having a weight of 283 mg and a purity of 96% with a yield of 42.9%.

The diclofenac prodrug of EX9, (((2-(2-hydroxyethoxy)ethoxy)carbonyl)oxy)methyl 2-(2-((2,6-dichlorophenyl)amino)phenyl)acetate with a chemical formula of

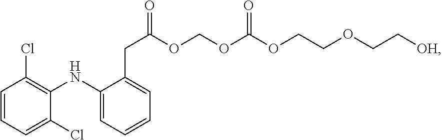 [SMILES: O=C(Cc1ccccc1Nc1c(Cl)cccc1Cl)OCOC(=O)OCCOCCO]

was subjected to NMR spectrometry using the procedures as described in EX1.

The diclofenac prodrug of EX9 was determined to have the following characteristics: ¹H NMR (CDCl₃, 500 MHz) δ 3.33 (br s, 3H), 3.54-3.56 (m, 1H), 3.66 (br s, 3H), 3.86 (s, 2H), 3.92 (br s, 1H), 4.16-4.23 (m, 2H), 4.16-4.23 (m, 2H), 5.78 (s, 2H), 6.54 (d, J=7.5 Hz, 1H), 6.58 (br s, 1H), 6.94-6.97 (m, 2H), 7.10-7.13 (m, 1H), 7.20-7.22 (m, 1H), 7.32 (d, J=8.5 Hz, 2H); ¹³C NMR (CDCl₃, 125 Hz) δ 38.02, 63.03, 69.24, 69.71, 82.56, 118.55, 122.29, 123.46, 124.24, 128.35, 128.89, 129.48, 131.03, 137.69, 142.65, 153.90, 170.97.

### Example 10 (EX10)

The procedures for preparing the diclofenac prodrug of EX10 include the following steps A to E.

In step A, a first solution containing 185 μL (2 mmol) of chloromethylchloroformate and 2 mL of dichloromethane was cooled to 0° C., and then mixed with a second solution containing 360 mg (3 mmol) of diethylene glycol monomethyl ether, 210 μL of pyridine, and 3 mL of dichloromethane to form a first mixture. Next, the first mixture was stirred at 0° C. for 2 hours and then at room temperature overnight, and then subjected to extraction followed by adding 30 mL of water and 10 mL of dichloromethane for extraction that is performed twice so as to form an organic layer. Thereafter, the organic layer thus collected was dried using anhydrous magnesium sulfate, followed by a filtration treatment so as to obtain a dehydrated organic layer. Afterwards, the dehydrated organic layer was subjected to a concentration treatment so as to obtain (((2-(2-methoxyethoxy)ethoxy)carbonyl)oxy)methyl chloride in the form of an intermediate oily product having a weight of 329 mg with a yield of 77%.

In step B, 458 mg (1.54 mmol) of diclofenac dissolved in 10 mL of acetone was mixed with 295 μL (1.64 mmol) of N,N-diisopropylethylamine, and then 329 mg (1.54 mmol) of the (((2-(2-methoxyethoxy)ethoxy)carbonyl)oxy)methyl chloride dissolved in 2 mL of acetone, and 231 mg (1.54 mmol) of sodium iodide were added in small portions, so as to form a second mixture.

In step C, the second mixture was heated and stirred at 65° C. for 24 hours, followed by a concentration treatment under reduced pressure to obtain a concentrated product.

In step D, the concentrated product was diluted by adding 30 mL of water, and then dichloromethane was added for twice extraction (20 mL each time) so as to form an organic layer. The organic layer thus collected was washed with 20 mL of brine, and then dried using anhydrous magnesium sulfate, followed by a filtration treatment so as to obtain a dehydrated organic layer.

In step E, the solvent in the dehydrated organic layer was removed under reduced pressure to obtain a crude product, which was diluted using 4.0 mL of acetonitrile/water solvent and then subjected to preparative reverse phase HPLC to determine purity thereof. The preparative reverse phase HPLC was performed under the following conditions: GL Science Inertsil ODS-3 column having a size of 30*250 mm, 10 μm; flow rate of 32.5 mL/minute; stepwise gradient elution from 55% acetonitrile/water to 75% acetonitrile/water with 0.1% of trifluoroacetic acid in water; UV detection under a wavelength of 254 nm. The thus collected peak-containing fractions were lyophilized to obtain a diclofenac prodrug of EX10 in the form of a colorless oil having a weight of 283 mg and a purity of 98% with a yield of 52.7%.

The diclofenac prodrug of EX10, 3-oxo-2,4,7,10-tetraoxaundecyl 2-(2-((2,6-dichlorophenyl)amino)phenyl)acetate with a chemical formula of

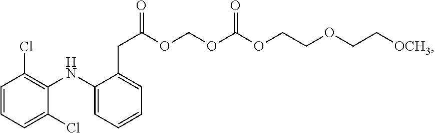 [SMILES: COCCOCCOC(=O)OCOC(=O)Cc1ccccc1Nc1c(Cl)cccc1Cl]

was subjected to NMR spectrometry using the procedures as described in EX1.

The diclofenac prodrug of EX10 was determined to have the following characteristics: ¹H NMR (CDCl₃, 500 MHz) δ 1.70 (s, 2H), 1.89-1.93 (m, 2H), 3.72 (t, J=6.0 Hz, 2H), 3.88 (s, 2H), 4.34 (t, J=6.5 Hz, 2H), 5.81 (s, 2H), 6.56 (d, J=8.0 Hz, 1H), 6.64 (bs, 1H), 6.96-6.99 (m, 2H), 7.12-7.15 (m, 2H), 7.12-7.15 (m, 1H), 7.23 (bs, 1H), 7.34 (d, J=8.0 Hz, 2H); ¹³C NMR (CDCl₃, 125 Hz) δ 31.41, 38.11, 58.84, 65.77, 82.38, (C, —OCH₂O—), 118.63, 122.29, 123.57, 124.13, 128.29, 129.44, 130.98, 137.75, 142.68, 154.05 (C, —OCOO—), 170.78 (C, —OCOCH₂—).

### Example 11 (EX11)

The procedures for preparing the diclofenac prodrug of EX11 include the following steps A to D.

In step A, a first solution containing 360 μL (4 mmol) of chloromethylchloroformate and 3 mL of acetonitrile was cooled to 0° C., and then a second solution containing 0.4 mL (4 mmol) of piperidine, 0.73 mL of N,N-diisopropylethylamine, and 6 mL of acetonitrile was added dropwise to the cooled first solution for 15 minutes, followed by stirring at 0° C. for 2 hours and then at room temperature overnight, thereby obtaining a first mixture.

In step B, a third solution containing 1.13 g (3.81 mmol) of diclofenac and 10 mL of acetonitrile were mixed with 0.73 mL (4 mmol) of N,N-diisopropylethylamine by stirring to dissolve the diclofenac, so as to form a second mixture. Next, the second mixture was added into the first mixture, followed by addition of 627 mg (4.18 mmol) of sodium iodide to form a third mixture.

In step C, the third mixture was heated and stirred at 70° C. for 3.5 hours, cooled to room temperature, and then subjected to an extraction treatment by mixing with 40 mL of ethyl acetate and 40 mL of water, so as to form an organic layer. The organic layer thus collected was washed with 20 mL of water twice, and then subjected to a concentration treatment under reduced pressure to obtain a concentrated product having a weight of 1.72 g.

In step D, the concentrated product was subjected to purification by silica gel chromatography with a stationary phase including silica gel (Merck) having a size of 40 μm to 63 μm, 60 Å, and a mobile phase including n-hexane and ethyl acetate that are present in a volume ratio ranging from 1:2 to 1:5, thereby obtaining a diclofenac prodrug of EX11 in the form of an oily product having a weight of 1.02 g with a yield of 61%.

The diclofenac prodrug of EX11, (2-(2-((2,6-dichlorophenyl)amino)phenyl)acetoxymethyl piperidine-1-carboxylate with a chemical formula of

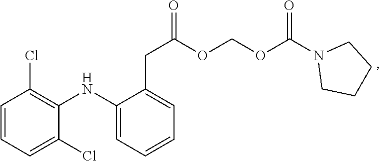 [SMILES: O=C(Cc1ccccc1Nc1c(Cl)cccc1Cl)OCOC(=O)N1CCCC1]

was subjected to NMR spectrometry using the procedures as described in EX1.

The diclofenac prodrug of EX11 was determined to have following characteristics: ¹H NMR (CDCl₃, 500 MHz) δ 1.66 (s, 2H), 1.83-1.86 (m, 4H), 3.30 (t, J=7.0, 2H), 3.40 (t, J=7.0, 2H), 3.87 (s, 2H), 5.82 (s, 2H), 6.55 (d, J=8.0 Hz, 1H). 6.75 (bs, 1H), 6.96-6.99 (m, 2H), 7.14-7.16 (m, 2H), 7.23-7.25 (m, 1H), 7.33 (d, J=8.5 Hz, 2H); ¹³C NMR (CDCl₃, 125 Hz) δ 24.82, 25.58, 38.36, 45.93, 46.31, 80.64, (C, —OCH₂O—), 118.45, 122.14, 123.87, 124.03, 128.12, 128.83, 129.47, 130.99, 137.81, 142.73, 152.89 (C, —OCOO—), 171.33 (C, —OCOCH₂—).

### Example 12 (EX12)

The procedures for preparing the diclofenac prodrug of EX12 include the following steps A to D.

In step A, a first solution containing 360 μL (4 mmol) of chloromethylchloroformate and 3 mL of acetonitrile was cooled to 0° C., and then a second solution containing 350 mg (4 mmol) of pyrrolidine, 210 μL of N,N-diisopropylethylamine, and 3 mL of acetonitrile was added dropwise to the cooled first solution for 12 minutes, followed by stirring at 0° C. for 2 hours and then at room temperature overnight, thereby obtaining a first mixture.

In step B, a third solution containing 1.13 g (3.81 mmol) of diclofenac and 10 mL of acetonitrile were mixed with 0.66 mL (3.81 mmol) of N,N-diisopropylethylamine by stirring to dissolve the diclofenac, so as to form a second mixture. Next, the second mixture was added into the first mixture, followed by addition of 627 mg (4.18 mmol) of sodium iodide to form a third mixture.

In step C, the third mixture was heated and stirred at a temperature ranging from 65° C. to 70° C. for 4 hours, cooled to room temperature, and then subjected to an extraction treatment by mixing with 30 mL of ethyl acetate and 30 mL of water, so as to form an organic layer. The organic layer thus collected was washed with 30 mL of water for three times, and then subjected to a concentration treatment under reduced pressure to obtain a concentrated product having a weight of 1.58 g.

In step D, the concentrated product was subjected to a recrystallization treatment using ethyl acetate and n-hexane that are present in a volume ratio of 1:1.5, thereby obtaining a diclofenac prodrug of EX12 in the form of a white solid powder having a weight of 1.11 g with a yield of 69%.

The diclofenac prodrug of EX12, (2-(2-((2,6-dichlorophenyl)amino)phenyl)acetoxy)methyl pyrrolidine-1-carboxylate with a chemical formula of

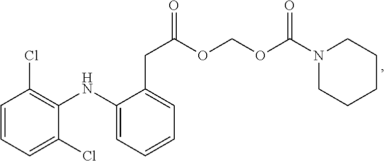 [SMILES: O=C(Cc1ccccc1Nc1c(Cl)cccc1Cl)OCOC(=O)N1CCCCC1]

was subjected to NMR spectrometry using the procedures as described in EX1.

The diclofenac prodrug of EX12 was determined to have the following characteristics: ¹H NMR (CDCl₃, 500 MHz) δ 1.43 (s, 2H), 1.44-1.60 (br s, 4H), 3.36-3.43 (m, 2H), 3.86 (s, 2H), 5.82 (s, 2H), 6.55 (d, J=8.0 Hz, 1H), 6.75 (bs, 1H), 6.95-7.11 (m, 2H), 7.14-7.16 (m, 2H), 7.23-7.25 (m, 1H), 7.33 (d, J=8.0 Hz, 2H); ¹³C NMR (CDCl₃, 125 Hz) δ 25.37, 38.34, 46.03, 80.79, (C, —OCH₂O—), 118.44, 122.15, 123.84, 124.04, 128.13, 128.83, 129.49, 130.98, 137.80, 142.72, 153.35 (C, —OCOO—), 171.37 (C, —OCOCH₂—).

### Example 13 (EX13)

The procedures for preparing the diclofenac prodrug of EX13 include the following steps A to D.

In step A, a first solution containing 360 μL (4 mmol) of chloromethylchloroformate and 3 mL of acetonitrile was cooled to 0° C., and then a second solution containing 0.46 mL (4 mmol) of 3-methylmorpholine, 0.86 mL of N,N-diisopropylethylamine, and 3 mL of acetonitrile was added dropwise to the cooled first solution for 15 minutes, followed by stirring at 0° C. for 2 hours and then at room temperature overnight, thereby obtaining a first mixture.

In step B, a third solution containing 1.13 g (3.81 mmol) of diclofenac and 10 mL of acetonitrile were mixed with 0.73 mL (4 mmol) of N,N-diisopropylethylamine by stirring to dissolve the diclofenac, so as to form a second mixture. Next, the second mixture was added into the first mixture, followed by addition of 627 mg (4.18 mmol) of sodium iodide to form a third mixture.

In step C, the third mixture was heated and stirred at a temperature of 70° C. for 4 hours, cooled to room temperature, and then subjected to an extraction treatment by mixing with 30 mL of ethyl acetate and 30 mL of water, so as to form an organic layer. The organic layer thus collected was washed with 30 mL of water for three times, and then subjected to a concentration treatment under reduced pressure to obtain a concentrated product having a weight of 1.87 g.

In step D, the concentrated product was subjected to purification by silica gel chromatography with a stationary phase including silica gel (Merck) having a size of 40 μm to 63 μm, 60 Å, and a mobile phase including ethyl acetate and n-hexane that are present in a volume ratio ranging from 1:1 to 1:5, thereby obtaining a diclofenac prodrug of EX13 in the form of an oily product having a weight of 1.18 g with a yield of 68%.

The diclofenac prodrug of EX13, (2-(2-((2,6-dichlorophenyl)amino)phenyl)acetoxy)methyl 2-methylmorpholine-4-carboxylate with a chemical formula of

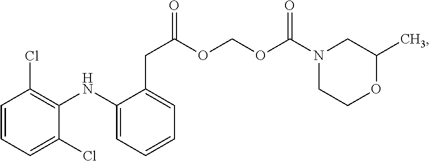 [SMILES: CC1CN(C(=O)OCOC(=O)Cc2ccccc2Nc2c(Cl)cccc2Cl)CCO1]

was subjected to NMR spectrometry using the procedures as described in EX1.

The diclofenac prodrug of EX13 was determined to have the following characteristics: ¹H NMR (CDCl₃, 500 MHz) δ 1.12-1.17 (m, 3H), 3.41-3.60 (m, 2H), 3.40-3.59 (m, 2H), 3.87 (s, 2H), 3.78-3.98 (m, 3H), 6.55 (d, J=8.0 Hz, 1H), 6.75 (bs, 1H), 6.96-6.99 (m, 2H), 7.13-7.16 (m, 2H), 7.24-7.26 (m, 1H), 7.34 (d, J=8.5 Hz, 2H); ¹³C NMR (CDCl₃, 125 Hz) δ 17.51, 38.31, 48.78, 49.03, 49.37, 71.56, 80.73 (C, —OCH₂O—), 118.44, 122.19, 123.69, 124.13, 128.22, 128.86, 129.47, 130.94, 137.74, 142.69, 153.09 (C, —OCOO—), 171.28 (C, —OCOCH₂—).

### Example 14 (EX14)

The procedures for preparing the diclofenac prodrug of EX14 include the following steps A to D.

In step A, a first solution containing 360 μL (4 mmol) of chloromethylchloroformate and 3 mL of acetonitrile was cooled to 0° C., and then a second solution containing 0.46 mL (4 mmol) of 2,6-dimethylmorpholine, 0.86 mL of N,N-diisopropylethylamine, and 3 mL of acetonitrile was added dropwise to the cooled first solution for 15 minutes, followed by stirring at 0° C. for 2 hours and then at room temperature overnight, thereby obtaining a first mixture.

In step B, a third solution containing 1.13 g (3.81 mmol) of diclofenac and 10 mL of acetonitrile were mixed with 0.73 mL (4 mmol) of N,N-diisopropylethylamine by stirring to dissolve the diclofenac, so as to form a second mixture. Next, the second mixture was added into the first mixture, followed by addition of 627 mg (4.18 mmol) of sodium iodide to form a third mixture.

In step C, the third mixture was heated and stirred at a temperature of 70° C. for 4 hours, cooled to room temperature, and then subjected to an extraction treatment by mixing with 30 mL of ethyl acetate and 30 mL of water, so as to form an organic layer. The organic layer thus collected was washed with 30 mL of water for three times, and then subjected to a concentration treatment under reduced pressure to obtain a concentrated product having a weight of 1.89 g.

In step D, the concentrated product was subjected to purification by silica gel chromatography with a stationary phase including silica gel (Merck) having a size of 40 μm to 63 μm, 60 Å, and a mobile phase including ethyl acetate and n-hexane that are present in a volume ratio ranging from 1:1 to 1:5, thereby obtaining a diclofenac prodrug of EX14 in the form of an oily product having a weight of 1.21 g with a yield of 68%.

The diclofenac prodrug of EX14, (2-(2-((2,6-dichlorophenyl)amino)phenyl)acetoxy)methyl-2,6-dimethylmorpholine-4-carboxylate with a chemical formula of

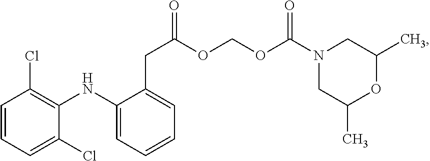 [SMILES: CC1CN(C(=O)OCOC(=O)Cc2ccccc2Nc2c(Cl)cccc2Cl)CC(C)O1]

was subjected to NMR spectrometry using the procedures as described in EX1.

The diclofenac prodrug of EX14 was determined to have the following characteristics: ¹H NMR (CDCl₃, 500 MHz) δ 1.12 (d, J=6.0 Hz, 3H), 1.18 (d, J=6 Hz, 3H), 2.48-2.53 (m, 2H), 3.41-3.56 (m, 2H), 3.81 (d, J=13 Hz, 1H), 3.87 (s, 2H), 3.87 (d, J=13 Hz, 1H), 5.84 (s, 2H), 6.56 (d, J=8.0 Hz, 1H), 6.75 (bs, 1H), 6.96-7.02 (m, 2H), 7.11-7.15 (m, 2H), 7.24-7.27 (m, 1H), 7.35 (d, J=8.5 Hz, 2H); ¹³C NMR (CDCl₃, 125 Hz) δ 18.62, 38.33, 49.01, 49.36, 71.46, 71.59, 80.71 (C, —OCH₂O—), 118.43, 122.19, 123.66, 124.16, 128.25, 128.88 (2C), 129.46, 130.99, 137.71, 142.69, (C, —OCOO—), 153.14, 171.36 (C, —OCOCH₂—).

### Example 15 (EX15)

The procedures for preparing the diclofenac prodrug of EX15 include the following steps A to D.

In step A, a first solution containing 360 μL (4 mmol) of 1-chloroethyl chloroformate and 3 mL of acetonitrile was cooled to 0° C., and then a second solution containing 0.38 mL (4 mmol) of morpholine, 0.86 mL of N,N-diisopropylethylamine, and 3 mL of acetonitrile was added dropwise to the cooled first solution for 15 minutes, followed by stirring at 0° C. for 2 hours and then at room temperature overnight, thereby obtaining a first mixture.

In step B, a third solution containing 1.13 g (3.81 mmol) of diclofenac and 10 mL of acetonitrile were mixed with 0.73 mL (4 mmol) of N,N-diisopropylethylamine by stirring to dissolve the diclofenac, so as to form a second mixture. Next, the second mixture was added into the first mixture, followed by addition of 625 mg (4.18 mmol) of sodium iodide to form a third mixture.

In step C, the third mixture was heated and stirred at a temperature of 70° C. for 4 hours, cooled to room temperature, and then subjected to an extraction treatment by mixing with 30 mL of ethyl acetate and 30 mL of water, so as to form an organic layer. The organic layer thus collected was washed with 30 mL of water for three times, and then subjected to a concentration treatment under reduced pressure to obtain a concentrated product having a weight of 1.89 g.

In step D, the concentrated product was subjected to purification by silica gel chromatography with a stationary phase including silica gel (Merck) having a size of 40 μm to 63 μm, 60 Å, and a mobile phase including ethyl acetate and n-hexane that are present in a volume ratio ranging from 1:3 to 1:5, thereby obtaining a brownish oily product having a weight of 0.98 g. Thereafter, the brown-colored oily product was subjected to a recrystallization treatment using ethyl acetate and n-hexane, thereby obtaining a diclofenac prodrug of EX15 in the form of a yellow solid having a weight of 0.80 g with a yield of 52%.

The diclofenac prodrug of EX15, (2-(2-((2,6-dichlorophenyl)amino)phenyl)acetoxy)methyl-2,6-dimethylmorpholine-4-carboxylate with a chemical formula of

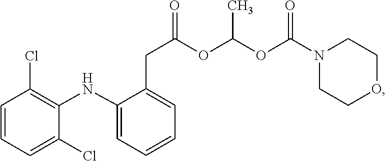 [SMILES: CC(OC(=O)Cc1ccccc1Nc1c(Cl)cccc1Cl)OC(=O)N1CCOCC1]

was subjected to NMR spectrometry using the procedures as described in EX1.

The diclofenac prodrug of EX15 was determined to have the following characteristics: ¹H NMR (CDCl₃, 500 MHz) δ 1.53 (d, J=5 Hz, 3H), 1.76 (br s, 2H), 3.38-3.56 (m, 2H), 3.81 (d, J=13 Hz, 1H), 3.85-3.45 (m, 4H), 3.46-3.65 (m, 4H), 3.83 (dd, J=14.5, 20.5 Hz, 2H), 6.56 (d, J=7.5 Hz, 1H), 6.71 (bs, 1H), 6.89 (q, J=6.0 Hz, 1H), 6.98-7.01 (m, 3H), 7.15-7.18 (m, 2H), 7.23-7.26 (m, 1H), 7.35 (d, J=8.5 Hz, 2H); ¹³C NMR (CDCl₃, 125 Hz) δ 19.79, 38.41, 43.80, 44.36, 66.29, 66.52, 90.56 (C, —OCH₂O—), 118.54, 122.18, 124.05, 128.07, 128.87 (2C), 129.43, 130.90, 137.86, 142.67, (C, —OCOO—), 152.91, 170.41 (C, —OCOCH₂—).

### Comparative Example (CE)

Sodium 2-{2-[(2,6-dichlorophenyl)amino]phenyl}acetate, hereinafter referred to as diclofenac sodium, which was purchased from Combi-Blocks Inc., directly serves as a Comparative Example.

### Property Evaluation

### 1. X-Ray Powder Diffraction Analysis

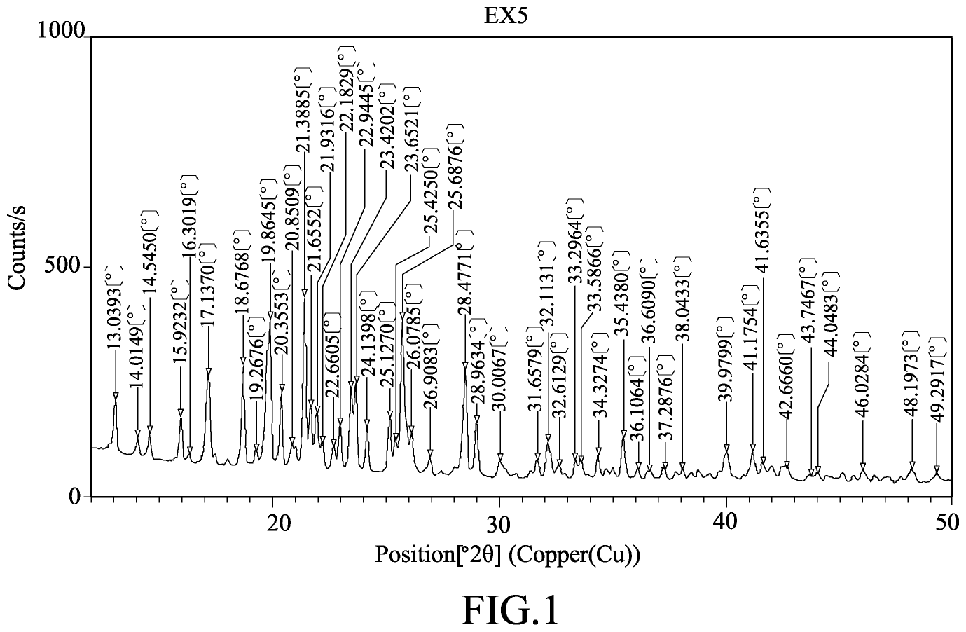

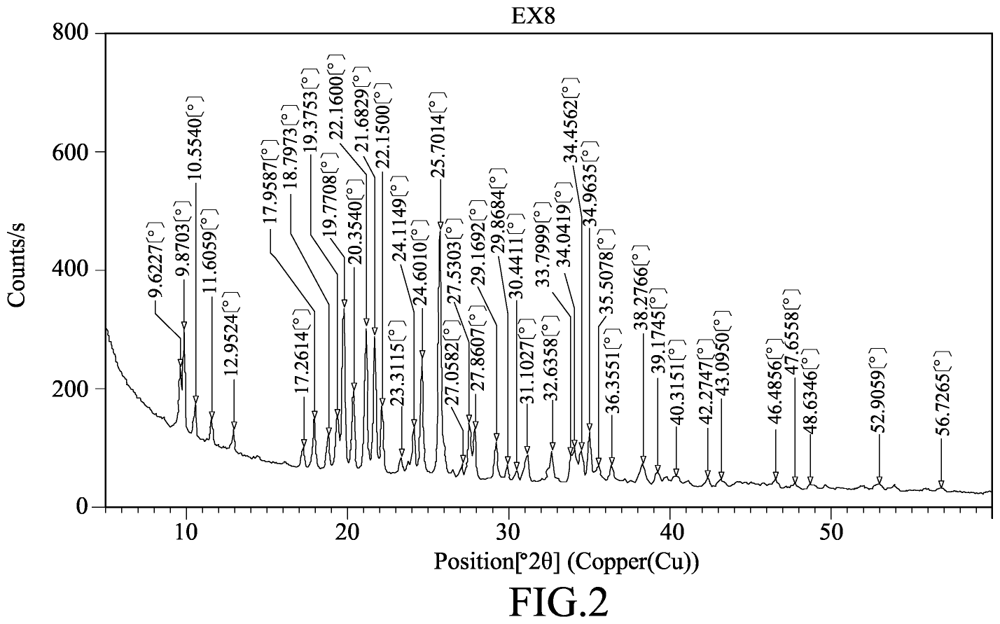

It should be noted that the diclofenac prodrugs of EX5 and EX8 each is in a solid crystalline form, and such diclofenac prodrugs were prepared using high polar solvents, e.g., methanol, ethanol. Isopropanol, ethyl acetate, acetonitrile, etc., and anti-solvents, e.g., hexane, heptane, isopropyl ether, etc. The diclofenac prodrugs of EX5 and EX8 were subjected to X-ray diffraction analysis using an X-ray diffractometer (Manufacturer: PANalytical; Model: Empyrean; scan range: 5° to 60°; scan step size: 0.04°) so as to determine crystallographic molecular structure thereof. The results are presented as plots of X-ray diffraction patterns, and were shown in FIGS. 1 and 2.

As shown in FIG. 1, the diclofenac prodrugs of EX5 showed sharp diffraction peaks in 20 degrees at 13.04, 15.92, 17.14, 18.68, 19.86, 20.35, 21.39, 21.65, 22.42, 23.65, 25.12, 25.68, 28.47 and 28.96.

As shown in FIG. 2, the diclofenac prodrugs of EX8 showed sharp diffraction peaks in 2θ degrees at 9.62, 9.87, 17.96, 19.37, 19.77, 20.35, 21.16, 21.68, 22.15, 24.11, 24.60, 25.70, 27.59, 27.86, 29.17 and 34.96.

### 2. Solubility and Hydrolysis Test

### A. Solubility Test Using Potassium Phosphate Buffer at pH 7.4

First, a respective one of the diclofenac prodrugs of EX5 to EX8 and EX12 was mixed with a potassium phosphate buffer (including 50 mM potassium phosphate, sodium chloride, and water; pH 7.4) and then placed in a constant temperature oscillator at 37° C. for 24 hours so as to dissolve the diclofenac prodrug, thereby obtaining a test sample. After that, the test sample was filtered through a filter having a pore size of 0.22 μm to obtain a filtrate. Next, the filtrate was mixed with acetonitrile in equal amounts, and then subjected to oscillation and centrifugation performed in sequence. The resultant supernatant fraction was subjected to HPLC with UV detection under a wavelength of 254 nm. The results are shown in Table 1 below.

TABLE 1

| Supernatant fraction | | Solubility |
|---|---|---|
| Diclofenac prodrug | Potassium | after |

| | Amount | phosphate | 24 hours |
|---|---|---|---|
| Form | (mg) | buffer (mL) | (μg/mL) |
| EX5 (amorphous) | 5 | 10 | 4.24 |
| EX5 (crystalline) | 5 | 10 | 0.06 |
| EX6 (crystalline) | 5 | 10 | 0.33 |
| EX7 (crystalline) | 5 | 10 | 268.53 |
| EX8 (crystalline) | 5 | 10 | 28.43 |
| EX12 (crystalline) | 5 | 10 | 0.69 |

The concentration of each of the diclofenac prodrugs of EX 5 to EX8 and EX12 was lower than limit of detection (LoD) of HPLC after dissolution for 24 hours. As shown in Table 1, each of the diclofenac prodrugs of EX5, EX6, EX8 and EX12 has a low solubility in the potassium phosphate buffer at pH 7.4, suggesting that the diclofenac prodrug of the present disclosure is slowly dissolved and released after administration, and thus, has a long half-life.

### B. Hydrolysis Test Using 80% Rat Serum

First, 1 mg of a respective one of the diclofenac prodrugs of EX5 and EX8 was mixed with 0.2 mL of the potassium phosphate buffer (including 50 mM potassium phosphate, sodium chloride, and water; pH 7.4) in a vial, followed by adding 0.8 mL of rat serum (purchased from BioLASCO Taiwan Co., Ltd., Model: CD(SD) Rat Serum) thereto. Next, the vial was placed in a constant temperature oscillator so as to dissolve the diclofenac prodrug at 37° C., and contents of the vial was taken out after 24 hours and 96 hours, thereby obtaining two test samples containing diclofenac. After that, each of the test samples was filtered through a filter having a pore size of 0.22 μm to obtain a filtrate. Next, the filtrate was mixed with acetonitrile in equal amounts, and then subjected to oscillation and centrifugation performed in sequence. The resultant supernatant fraction was subjected to HPLC with UV detection under a wavelength of 254 nm. The results are shown in Table 2 below.

TABLE 2

| Supernatant fraction | | | | Solubility | Solubility |
|---|---|---|---|---|---|
| Diclofenac prodrug | | Potassium | Rat | after | after |
| | Amount | phosphate | serum | 24 hours | 96 hours |
| Form | (mg) | buffer (mL) | (mg) | (μg/mL) | (μg/mL) |
| EX5 | 1 | 0.2 | 0.8 | 0.06 | 102.99 |
| (crystalline) | | | | | |
| EX8 | 1 | 0.2 | 0.8 | 28.43 | 126.93 |
| (crystalline) | | | | | |

As shown in Table 2, each of the diclofenac prodrugs of EX5 and EX8 has a low solubility in the rat serum, suggesting that the diclofenac prodrug of the present disclosure is slowly dissolved and released after administration, and thus, has a long half-life.

### 3. Determination of Particle Size

The solid particles of the diclofenac prodrugs of EX5 and EX8 were subjected to grinding in order to be formulated into a soluble liquid suspension suitable for intraarticular injection. In general, first, the solid particles of diclofenac prodrugs and deionized water were mixed to form a liquid suspension. Next, the liquid suspension was ground using a homogenizer and then subjected to oscillation treatment to obtain a test sample. Then, the test sample was subjected to determination of particle size distribution. Afterwards, the test sample includes solid particles having a size ranging from 20 μm to 30 μm was centrifuged and lyophilized, followed by determination of purity using HLPC. It should be noted that solid particles having a relatively large particle size, i.e., ranging from 60 μm to 90 μm, are required to be subjected to a pre-grinding treatment using a mortar in the presence of a grinding medium which includes a dispersant (e.g., Pluronic® F-68, Tween 20, Tween 80, and Triton X-100), water, and physiological saline.

To be specific, 100 mg of the diclofenac prodrug of EX5 having an initial particle size of about 60 μm was manually ground using a mortar, and then mixed with 2.0 mL of an aqueous solution containing 0.125 wt % of Tween 20 and deionized water, so as to obtain a slurry. Next, the slurry was ground using a homogenizer and subjected to oscillation at a rotation speed of 29 K rpm for 15 minutes, followed by determination of particle size D90 using a particle size analyzer (Manufacturer: Beckman Coulter; Model: Multisizer™ 3) for 3 times, each time with a volume of 20 μL. The thus determined average particle size D90 of the diclofenac prodrug of EX5 was 8.4 μm.

Meanwhile, 102 mg of the diclofenac prodrug of EX8 having an initial particle size of about 60 μm was manually ground using a mortar, and then mixed with 2.0 mL of an aqueous solution containing 0.125 wt % of Tween 20 and saline, so as to obtain a slurry. Next, the slurry was ground using a homogenizer and subjected to oscillation at a rotation speed of 29 K rpm for 15 minutes, and then placed into an ice bath for 1 minute in every 5 minutes for a total time period of 33 minutes, followed by determination of particle size D90 using a particle size analyzer (Manufacturer: Beckman Coulter; Model: Multisizer™ 3) for 3 times, each time with a volume of 20 μL. The thus determined average particle size D90 of the diclofenac prodrug of EX8 was 18.2 μm.

### 4. Pharmacokinetic Study

Three liquid suspensions, each having a concentration of 70 mmol/mL, were respectively prepared using the diclofenac sodium of CE, the diclofenac prodrug of EX5 which was ground with an average particle size D90 of 8.4 μm, and the diclofenac prodrug of EX8 which was ground with an average particle size D90 of 18.2 μm, to be used in a pharmacokinetic study described hereinafter.

For preparing the liquid suspension containing the diclofenac sodium of CE, 3 mL of a hyaluronic acid solution containing 0.1 wt % of hyaluronic acid (molecular weight: 2000 kDa, purchased from Aldrich) and sterile saline (purchased from Taiwan Biotech Co., Ltd.) was added to 66 mg of the diclofenac sodium of CE, followed by an oscillation treatment.

For preparing the liquid suspension containing the diclofenac prodrug of EX5, 3 mL of a hyaluronic acid solution containing 0.1 wt % of hyaluronic acid (Aldrich) and sterile saline (Taiwan Biotech Co., Ltd.) was added to 92 mg of the diclofenac prodrug of EX5, followed by an oscillation treatment.

For preparing the liquid suspension containing the diclofenac prodrug of EX8, 3 mL of a hyaluronic acid solution containing 0.1 wt % of hyaluronic acid (Aldrich) and sterile saline (Taiwan Biotech Co., Ltd.) was added to 92 mg of the diclofenac prodrug of EX8, followed by an oscillation treatment.

Experimental subjects used in the pharmacokinetic study were male Lewis rats (10 to 12 weeks old), which were housed in an animal room with an independent air conditioning system under the following laboratory conditions: an alternating 12-hour light and 12-hour dark cycle, a temperature maintained at 25° C.±2° C., and a relative humidity maintained at 40% to 70%. The rats were provided with water and fed ad libitum. All experimental procedures involving the experimental rats were in compliance with the legal provision of the Animal Protection Act of Taiwan, and were carried out according to the guidelines of the Animal Care Committee of the Council of Agriculture, Taiwan.

The rats were randomly divided into three groups, namely, Experimental group 1 (EG1), Experimental group 2 (EG2), and Comparative group (CG), in which the number of rats in each of the EG1, EG2 and CG was 3.

It should be noted that each of aforesaid liquid suspensions was prepared on the same day, and was administered to the rats in each group within 4 hours.

To be specific, the rats in the EG1 were intraarticularly administered with the liquid suspension containing the diclofenac prodrug of EX5, the rats in the EG2 were intraarticularly administered with the liquid suspension containing the diclofenac prodrug of EX8, and the rats in the CG were intraarticularly administered with the liquid suspension containing the diclofenac sodium of CE. The intraarticular administration was performed by injecting a single dose of the respective liquid suspension into the joint cavity of the rats using a 29G needle. For the rats in the EG1, EG2 and CG, samples were collected from the blood, synovial tissues and meniscus of each rat on the 1^{st} day, 3^{rd} day, 7^{th} day, 14^{th} day and 28^{th} day post-administration and processed to obtain plasma samples, meniscus samples, and synovial tissue samples, respectively, followed by measurement of concentrations of diclofenac (i.e., the parent drug) therein.

The thus collected plasma from each rat was evenly mixed with a diluent (containing acetonitrile and 0.1 wt % of formic acid) in a ratio of 1:3, and with 50 ng/mL of glybenzcyclamide (serving as internal standard) using an oscillator (Brand: ThermoMixer) at constant temperature, followed by centrifugation so as to obtain a supernatant fraction that serves as the plasma sample.

The thus collected meniscus from each rat was mixed with a diluent (containing 0.1% formic acid, 20% acetonitrile and phosphate-buffered saline) in a ratio of 1:10, and then ground using a homogenizer (Manufacturer: Qiagen; Model: TissueLyser), followed by centrifugation at a speed of 12000 rpm for 5 minutes to obtain a first supernatant fraction. Afterwards, the first supernatant fraction was evenly mixed with a diluent (containing acetonitrile and 0.1 wt % of formic acid) in a ratio of 1:3, and with 50 ng/mL of glybenzcyclamide (serving as internal standard) using an oscillator (Brand: ThermoMixer) at constant temperature, followed by centrifugation so as to obtain a second supernatant fraction that serves as the meniscus sample.

The thus collected synovial tissue from each rat was mixed with a diluent (containing 0.1% formic acid, 20% acetonitrile and phosphate-buffered saline) in a ratio of 1:10, and then ground using a homogenizer (Manufacturer: Qiagen; Model: TissueLyser), followed by centrifugation at a speed of 12000 rpm for 5 minutes to obtain a first supernatant fraction. Afterwards, the first supernatant fraction was evenly mixed with a diluent (containing acetonitrile and 0.1 wt % of formic acid) in a ratio of 1:3, and with 50 ng/mL of glybenzcyclamide (serving as internal standard) using an oscillator (Brand: ThermoMixer) at constant temperature, followed by centrifugation so as to obtain a second supernatant fraction that serves as the synovial tissue sample.

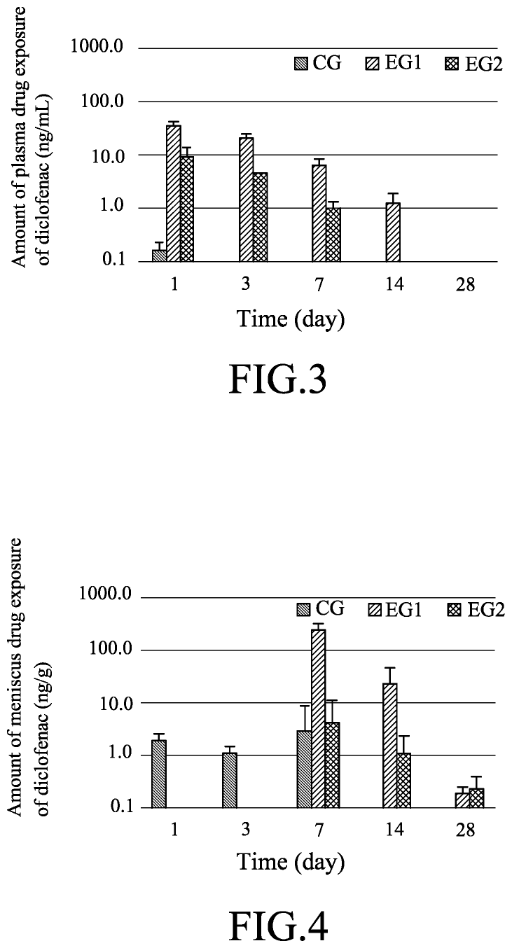

Each of the plasma samples, the meniscus samples and the synovial tissue samples was subjected to liquid chromatography-tandem mass spectrometry (LC-MS/MS) so as to determine the concentrations of diclofenac (i.e., parent drug) therein. The LC-MS/MS was performed using ACQUITY UPLC BEH C18 column (size: 1.7 μm; 2.1×50 mm), the first eluent was an aqueous formic acid solution including 0.1 wt % of formic acid and water, the second eluent was a formic acid solution including 0.1 wt % of formic acid and acetonitrile, and the conditions for elution were as follows: the flow rate of each of the first and second eluents was 0.4 mL/min; 50% of the first eluent and 50% of the second eluent were used at the 0^{th} minute of elution; 50% of the first eluent and 50% of the second eluent were used at the 0.5^{th} minute of elution; only the first eluent was used at the 6^{th} minute; only the first eluent was used at the 7^{t}h minute; 50% of the first eluent and 50% of the were used at the 7.5^{th} minute; and 50% of the first eluent and 50% of the second eluent were used at the 8.5^{th} minute. The results are shown in FIGS. 3 to 5.

As shown in FIG. 3, the amount of drug exposure of diclofenac (i.e., the parent drug) detected in the plasma of the rats of EG1 and EG2 on the 1^{st} day were 40.6±3.2 ng/mL and 9.7±4.8 ng/mL, respectively, and the concentrations of diclofenac decrease with increasing number of days post-administration in these groups. In contrast, the concentration of diclofenac detected in the plasma of the rats of EG on the first day was only 0.2±0.1 ng/mL. The concentrations of diclofenac detected in the plasma of the rats of EG1 and EG2 on the 1^{st}, 3^{rd}, 7^{th}, 14th and 28th days were all within the limit of quantification (LoQ). Note that the LoQs for the diclofenac of CE, the diclofenac prodrug of EX5 and the diclofenac prodrug of EX8 in each of the plasma samples were 0.05 ng/mL, 0.05 ng/mL, and 0.05 ng/mL, respectively.

As shown in FIG. 4, the amount of drug exposure of diclofenac (i.e., the parent drug) detected in the meniscus of the rats of EG1 and EG2 were highest at the 7^{th} day post-administration compared to other time points post-administration, i.e., 2484.2±935.9 ng/g and 42.9±67.5 ng/g, respectively, and the amounts of drug exposure of diclofenac decrease with increasing number of days post-administration in these groups. In contrast, the amounts of drug exposure of diclofenac detected in the meniscus of the rats of EG on the 7th day was only 30.1±55.9 ng/g. In addition, on the 7^{th} day post-administration, the maximal values of the amounts of drug exposure of diclofenac detected in the meniscus of the rats of EG1 and EG2 reached 70283.3±31746.4 ng/g and 4.1±1.4 ng/g, respectively. Note that the LoQs for the diclofenac of CE, the diclofenac prodrug of EX5 and the diclofenac prodrug of EX8 in each of the meniscus samples were 1.0 ng/g, 0.5 ng/g, and 0.5 ng/g, respectively.

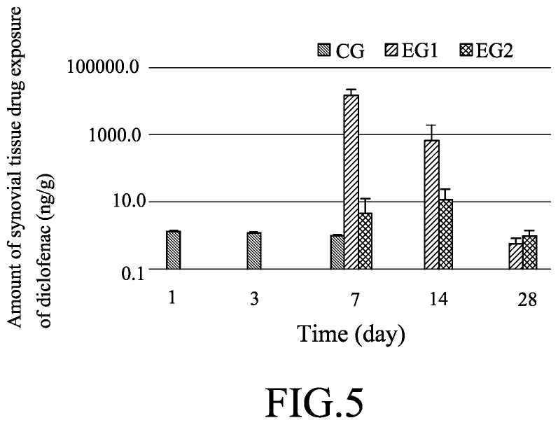

As shown in FIG. 5, the amount of drug exposure of diclofenac (i.e., the parent drug) detected in the synovial tissue of the rats of EG1 and EG2 were highest at the 7^{th} day post-administration compared to other time points post-administration, i.e., 16236.7±10516.7 ng/g and 13.3±13.1 ng/g, respectively, and the amounts of drug exposure of diclofenac decrease with increasing number of days post-administration in these groups. In contrast, the amounts of drug exposure of diclofenac detected in the synovial tissue of the rats of EG on the 7th day was only 1.3 ng/g. In addition, on the 7^{th} day post-administration, the maximal values of the amounts of drug exposure of diclofenac detected in the synovial tissue of the rats of EG1 and EG2 reached 2584666.7±1387275.7 ng/g and 2.1±0.8 ng/g, respectively. Note that the LoQs for the diclofenac of CE, the diclofenac prodrug of EX5 and the diclofenac prodrug of EX8 in each of the synovial tissue samples were 0.5 ng/g, 0.5 ng/g, and 0.5 ng/g, respectively.

Taken together, these results demonstrate that the diclofenac prodrug of the present disclosure, when administered by intraarticular injection, can be slowly dissolved and hydrolyzed, so as to slowly release diclofenac in the joint cavity, and hence, is expected to be useful for alleviating arthritis.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A diclofenac prodrug, which is represented by formula (I), 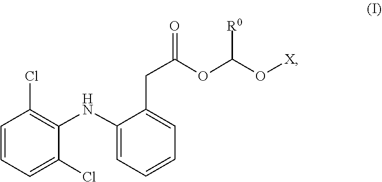
wherein in formula (I),
R⁰ represents hydrogen, a straight chain alkyl group or a branched chain alkyl group,
X represents R¹, 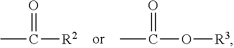
R¹ and R² are independently selected from the group consisting of a straight chain alkyl group, a branched chain alkyl group, a substituted heterocyclic group, an unsubstituted heterocyclic group, and 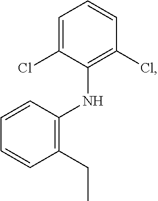 [SMILES: CCc1ccccc1Nc1c(Cl)cccc1Cl]
and
R³ is selected from the group consisting of a substituted phenyl group, an unsubstituted phenyl group, a substituted cycloalkyl group, an unsubstituted cycloalkyl group, a substituted heterocyclic group, an unsubstituted heterocyclic group, an alkyl with heterocyclic group, an alkyl with hydroxyl group, and (R³¹O)ₙ—R³²,
wherein R³¹ represents an alkylene group, R³² represents hydrogen or an alkyl group, and n is an integer greater than 2.

2. The diclofenac prodrug as claimed in claim 1, wherein R⁰ represents hydrogen, and X represents 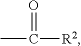
R² being selected from the group consisting of a straight chain alkyl group, a substituted heterocyclic group, an unsubstituted heterocyclic group, and

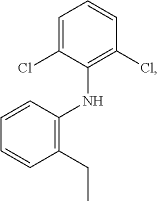 [SMILES: CCc1ccccc1Nc1c(Cl)cccc1Cl]

3. The diclofenac prodrug as claimed in claim 1, wherein R⁰ represents hydrogen, and X represents 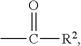
R² being selected from the group consisting of —CH₂CH₂CH₃, CH₃,

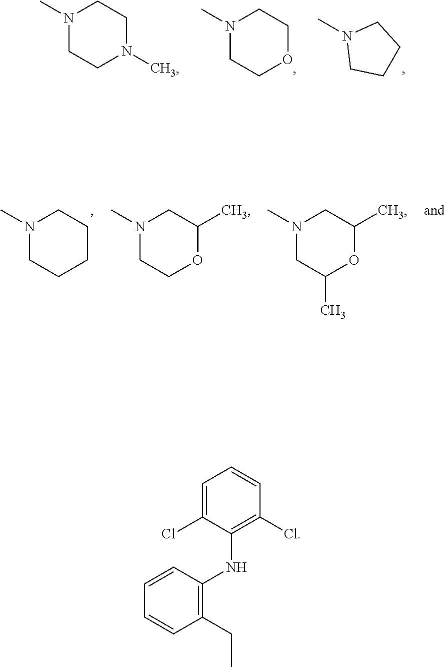

4. The diclofenac prodrug as claimed in claim 1, wherein R⁰ represents hydrogen, and X represents 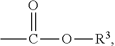
R³ being selected from the group consisting of 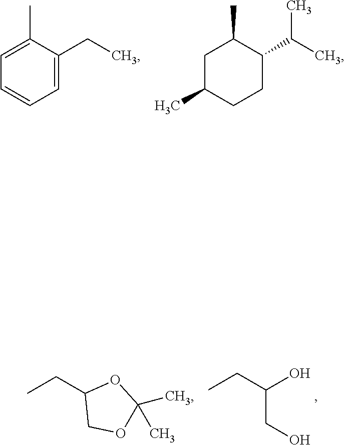
—CH₂CH₂OH, —CH₂CH₂CH₂OH, —CH₂CH₂OCH₂CH₂OH, and CH₂CH₂OCH₂CH₂OCH₃.

5. The diclofenac prodrug as claimed in claim 1, wherein 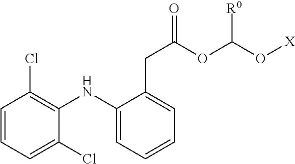
is 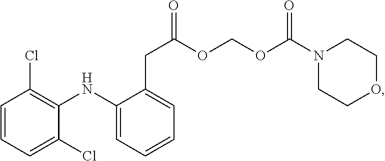 [SMILES: O=C(Cc1ccccc1Nc1c(Cl)cccc1Cl)OCOC(=O)N1CCOCC1]
which is in a crystalline form, and an X-ray diffraction pattern of 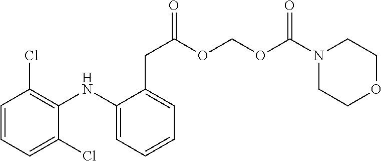 [SMILES: O=C(Cc1ccccc1Nc1c(Cl)cccc1Cl)OCOC(=O)N1CCOCC1]
shows characteristics peaks in 2θ degrees at 13.04, 15.92, 17.14, 18.68, 19.86, 20.35, 21.39, 21.65, 22.42, 23.65, 25.12, 25.68, 28.47 and 28.96.

6. The diclofenac prodrug as claimed in claim 1, wherein, 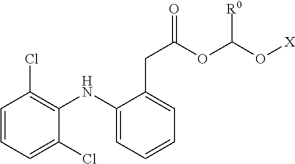
is 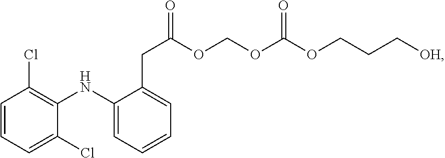 [SMILES: O=C(Cc1ccccc1Nc1c(Cl)cccc1Cl)OCOC(=O)OCCCO]
which is in a crystalline form, and an X-ray diffraction pattern of 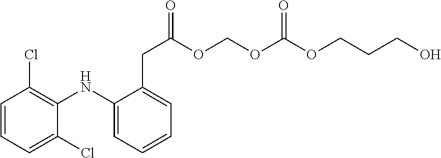 [SMILES: O=C(Cc1ccccc1Nc1c(Cl)cccc1Cl)OCOC(=O)OCCCO]
shows characteristics peaks in 2θ degrees at 9.62, 9.87, 17.96, 19.37, 19.77, 20.35, 21.16, 21.68, 22.15, 24.11, 24.60, 25.70, 27.59, 27.86, 29.17 and 34.96.

7. A method for alleviating arthritis, comprising administering to a subject in need thereof a diclofenac prodrug as claimed in claim 1.

8. The method as claimed in claim 7, wherein the diclofenac prodrug is in a dosage form for intraarticular administration.

9. The method as claimed in claim 7, wherein the arthritis is selected from the group consisting of osteoarthritis, rheumatoid arthritis, gouty arthritis, psoriatic arthritis, infectious arthritis, and combinations thereof.
